(19) United States
(12) Patent Application Publication
(10) Pub. No.: US 2026/0090749 A1
(43) Pub. Date: Apr. 2, 2026

(54) Devices and Methods for Fatigue Detection

(71) Applicant: The Board of Trustees of the Leland Stanford Junior University, Stanford, CA (US)

(72) Inventors: Peter Luke Santa Maria, Stanford, CA (US); Kristen K. Steenerson, Stanford, CA (US); Danyang Fan, Stanford, CA (US); Stephen Kargotich, Stanford, CA (US); Bradley C. Liang, Stanford, CA (US); Julia Persche, Stanford, CA (US)

(21) Appl. No.: 19/111,489

(22) PCT Filed: Sep. 14, 2023
(86) PCT No.: PCT/US2023/032773

§ 371 Date: Mar. 13, 2025

Related U.S. Application Data
(60) Provisional application No. 63/407,564, filed on Sep. 16, 2022.

(51) Int. Cl. A61B 5/16; A61B 5/00; A61B 5/11; A61B 5/296; A61B 5/397

(52) U.S. Cl. CPC A61B 5/165; A61B 5/0004; A61B 5/1103; A61B 5/296; A61B 5/397; A61B 5/6803; A61B 5/7264

(57) ABSTRACT

A method for assessing fatigue of a subject is provided. The method includes providing a device comprising a first electrode, a second electrode, and a circuit operably coupled to the first and second electrodes, the first and second electrodes configured to detect an electrical activity associated with an eye blink of a subject, and the circuit is configured to process a signal from the first electrode and the second electrode; determining a blink event based on the electrical activity; and assessing a degree of fatigue of the subject, based on the blink event. A headset including circuitry, such as a processor and a memory storing instructions to cause the headset to perform the above method are also provided. Also provided are systems and kits that include the devices. The methods, devices, headsets, systems and kits find use in a variety of different applications.

### CROSS-REFERENCE TO RELATED APPLICATIONS

This application claims priority to the filing date of U.S. Provisional patent application Ser. No. 63/407,564, filed Sep. 16, 2022, the disclosure of which application is incorporated herein by reference.

### TECHNICAL FIELD

The subject matter described herein relates to methods for assessing fatigue of a subject using a device to detect an electrical activity associated with an eye blink of a subject.

### BACKGROUND

People are susceptible to fatigue due to lifestyle and work requirements. Sleep loss, irregular working schedule (e.g., shift work), and extended periods of time spent on a monotonous or repetitive task are among common factors leading to fatigue and drowsiness, with consequential cognitive deficits. Fatigue can have serious consequences for people's health and safety and can negatively affect performance and quality of life.

For example, the operation of vehicles, on land, air, and water, requires attention by the vehicle operator. Drowsiness and/or fatigue can detract from the attention required to operate the vehicle safely. Methods for predicting, for example, whether the operator of a vehicle is likely to become fatigued, determining whether the operator of a vehicle is fatigued, and/or monitoring whether the operator of a vehicle is becoming fatigued are needed.

Fatigue is a problem in other areas, such as in the workplace. Fatigue contributes to reduced efficiency at work, and depending on the work, can pose a safety concern for the worker and for others working with or interacting with or depending on the fatigued person.

### BRIEF SUMMARY

The following aspects and embodiments thereof described and illustrated below are meant to be exemplary and illustrative, not limiting in scope.

In one aspect, a method for assessing fatigue of a subject is provided. The method comprises providing a device comprising a first electrode, a second electrode, and a circuit operably coupled to the first and second electrodes, the first and second electrodes configured to detect an electrical activity associated with an eye blink of a subject, and the circuit is configured to process a signal from the first electrode and the second electrode, determining a blink event based on the electrical activity, and assessing a degree of fatigue of the subject based on the blink event.

In another aspect, a method for predicting or determining fatigue in a subject is provided. The method comprises sensing an electrical activity associated with an eye blink of a subject, the sensing performed by a first electrode at a first facial position and a second electrode at a second facial position, where the first facial position and the second facial position are positioned to sense electrical activity of a single eye on the subject; and evaluating an electrical signal from the electrical activity sensed by the first electrode and the second electrode to detect an eye blink event that is correlated with a presence or an absence of fatigue in the subject.

In another aspect, a computer-implemented method for determining fatigue of a subject is provided. The method comprises obtaining a signal from an electrical activity associated with an eye blink of a subject; analyzing, using a processor, the signal to determine or measure a blink event for the subject; determining, using the processor, a fatigue indicator based on the blink event, and based on the fatigue indicator, performing one or more of (i) continuing said obtaining, analyzing and determining; (ii) transmitting the fatigue indicator to the subject or another person; or (iii) providing a stimulus to the subject.

In another aspect, a method to assess fatigue in real-time is provided. The method comprises providing to a subject a system comprised of (i) a wearable device comprising a first electrode, a second electrode and circuitry operably connected to the first and second electrodes, the first and second electrodes configured to detect electrical activity associated with an eye blink of a subject, and the circuitry is configured to process a signal from the first and second electrodes and (ii) a software application comprising an algorithm for evaluating the signal, and instructing the subject to (i) install the software application on a computing device, (ii) place the wearable device on their person proximal to an eye, and (iii) using the software application on the computing device, initiate a baseline assessment of eye movement and eye blink. The method further comprises instructing the subject to begin a planned activity and/or to continue to wear the wearable device during the electrical activity, wherein the wearable device senses electrical activity associated with eye blink, transmits signal corresponding to the electrical activity to a processor on the computing device for processing by an algorithm in the software application, to determine or measure a blink event to assess fatigue.

In another aspect, a headset is provided. The headset comprises an eyepiece configured to provide an image to a user; a facial interface configured to fix the eyepiece to face of the user; and a blink detector mounted on the facial interface. The blink detector comprises a first electrode and a second electrode mounted on the facial interface and configured to contact a skin of the user to detect an electrical activity associated with an eye blink of the user; and a circuit operably coupled to the first and second electrodes, configured to process a signal from the first and the second electrodes and identify a blink event by the user based on the electrical activity.

In addition to the exemplary aspects and embodiments described above, further aspects and embodiments will become apparent by reference to the drawings and by study of the following descriptions.

Additional embodiments of the present devices, systems, methods and kits will be apparent from the following description, drawings, examples, and claims. As can be appreciated from the foregoing and following description, each and every feature described herein, and each and every combination of two or more of such features, is included within the scope of the present disclosure provided that the features included in such a combination are not mutually inconsistent. In addition, any feature or combination of features may be specifically excluded from any embodiment of the present disclosure. Additional aspects and advantages of the present disclosure are set forth in the following description and claims, particularly when considered in conjunction with the accompanying examples and drawings.

### BRIEF DESCRIPTION OF THE DRAWINGS

FIGS. 1A-1B depict embodiments of a sensor for capturing and identifying electrical activity associated with an eye blink events, and, in some cases, monocular corneo-retinal potential.

FIG. 2 illustrates a system for detecting eye blinks from a subject including a wearable device and a software application installable onto or installed on a mobile device, according to one embodiment.

FIGS. 3A-3G illustrate goggles including a sensor for detecting eye blinks from a user, according to some embodiments.

FIGS. 4A-4C illustrate a processing algorithm and some exemplary signals obtained from a blink detector and used for assessing a fatigue factor of a subject, according to some embodiments.

FIG. 5 provides a depiction of a headset including a sensor for detecting an eye blink of the user, according to some embodiments.

FIG. 6 illustrates a helmet including a sensor for detecting eye blinks from a user, according to some embodiments.

FIGS. 7A-7C illustrate a headset including a sensor for detecting eye blinks from a user, according to some embodiments.

FIG. 8 is a flow chart illustrating steps in a method for determining a fatigue indicator of a subject, based on detection of eye blinks of the subject, according to some embodiments.

FIG. 9 is a flow chart illustrating steps in a method for determining a fatigue indicator of a subject, based on detection of eye blinks of the subject, according to some embodiments.

FIG. 10 is a flow chart illustrating steps in a method for determining fatigue in a subject, based on detection of eye blinks of the subject, according to some embodiments.

FIG. 11 is a block diagram illustrating a computer system configured to perform a method as in FIGS. 8-10, according to some embodiments.

In the figures, elements having the same or similar reference numerals have the same or similar features, unless explicitly stated otherwise.

### DETAILED DESCRIPTION

### I. Definitions

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art. It is also to be understood that the terminology used herein is for the purpose of describing particular embodiments only, and is not intended to be limiting, since the scope of the present invention will be limited only by the appended claims.

Where a range of values is provided, it is intended that each intervening value between the upper and lower limit of that range and any other stated or intervening value in that stated range is encompassed within the disclosure. For example, if a range of 1 μm to 8 μm is stated, it is intended that 2 μm, 3 μm. 4 μm, 5 μm, 6 μm, and 7 μm are also explicitly disclosed, as well as the range of values greater than or equal to 1 μm and the range of values less than or equal to 8 μm.

The singular forms “a,” “an,” and “the” include plural referents unless the context clearly dictates otherwise. Thus, for example, reference to a “polymer” includes a single polymer as well as two or more of the same or different polymers, reference to an “excipient” includes a single excipient as well as two or more of the same or different excipients, and the like.

The word “about” when immediately preceding a numerical value means a range of plus or minus 10% of that value, e.g., “about 50” means 45 to 55, “about 25,000” means 22,500 to 27,500, etc., unless the context of the disclosure indicates otherwise, or is inconsistent with such an interpretation. For example, in a list of numerical values such as “about 49, about 50, about 55,” “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein.

The compositions of the present disclosure can include, consist essentially of, or consist of, the components disclosed.

By reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by reserving the right to proviso out or exclude any individual substituents, analogs, compounds, ligands, structures, or groups thereof, or any members of a claimed group, less than the full measure of this disclosure can be claimed for any reason.

Throughout this disclosure, various patents, patent applications and publications are referenced. The disclosures of these patents, patent applications and publications in their entireties are incorporated into this disclosure by reference in order to more fully describe the state of the art as known to those skilled therein as of the date of this disclosure. This disclosure will govern in the instance that there is any inconsistency between the patents, patent applications, and publications cited in this disclosure.

For convenience, certain terms employed in the specification, examples, and claims are collected here. Unless defined otherwise, all technical and scientific terms used in this disclosure have the same meanings as commonly understood by one of ordinary skill in the art to which this disclosure belongs.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### II. Methods

In an embodiment, methods for assessing fatigue in a subject are provided. Fatigue refers to any state of reduced mental or physical performance capability resulting from sleep loss, extended wakefulness, circadian phase, and/or workload (mental and/or physical activity) that can impair a person's alertness and ability to perform safety-related operational duties. Thus, in the embodiment, fatigue is not distinguished from drowsiness. In other embodiments, methods for assessing mental fatigue in a subject are provided, where mental fatigue is distinguished from drowsiness or sleepiness, the latter due to circadian rhythm disruptions, sleep loss, and/or time awake and the former due to time-on-task and cognitive workload. The description infra refers to fatigue and intends to encompass drowsiness; however, it can be appreciated that the description equally refers to mental fatigue.

Fatigue is associated with an increased eye blink frequency and blink duration. Fatigue can also be detected by blink amplitude, e.g., the distance traveled by the subject's eyelid from maximum opening to minimum opening. A non-intrusive assessment of fatigue or drowsiness is provided by determining an eye blink event, such as blink frequency, frequency pattern, blink duration, blink bursts, blink phase (e.g., how long the lid takes to open and how long it stays closed during a blink) and/or blink amplitude with an electronic device that is non-intrusive to the subject and wearable by the subject during periods of activity. The electronic device can be mounted on an augmented reality headset (e.g., goggles, helmet, visor, and the like) worn by a subject, such as a pilot or driver, and can be used to provide feedback about the subject's, such as the pilot's or driver's, status. The feedback may include alerting the subject of their condition and providing certain actions and measures to keep the subject alert and awake, or communicating the subject's condition to a command center, or an emergency hub or control, to assess the situation.

“Fatigue” as used herein includes, but is not limited to, a feeling of weariness, tiredness, and/or lack of energy. A person feeling fatigued is often drowsy. In embodiments, fatigue exemplified by weariness, tiredness, lack of energy, and/or drowsiness may or may not be associated with “mental fatigue.” Mental fatigue may be distinct from fatigue by excluding the feeling of tiredness or drowsiness.

In one aspect, a method for assessing fatigue of a subject is provided. The method includes providing a device including a first electrode, a second electrode, and circuitry operably coupled to the first and second electrodes, the first and second electrodes configured to detect a voluntary eye blink of the subject. In some embodiments, a distinction between a voluntary blink and a reflex blink provoked by an external stimulus, is desirable, as, unlike voluntary blinks, reflex blinks may not be related to a subject's fatigue.

Embodiments as disclosed herein provide an eye blink detector to determine eye blink information for an individual, i.e., a subject. In addition, a processor receiving signals from the eye blink detector determines an associated value or values, such as eye blink rate, eye blink duration, eye blink frequency, eye blink amplitude and/or patterns of eye blinks. Accordingly, one or more mental states of the individual are inferred based on the eye blink information, including drowsiness and fatigue of the subject.

In some embodiments, the method further includes manipulating aspects of a subject's environment, such as a screen on a headset or helmet or even aspects of a vehicle or other machinery operated by the individual, based on the mental states inferred from an eye blink measurement. Mental states of interest may include one or more of attention, concentration, boredom, fatigue, or cognitive load. For example, a higher blink-rate frequency may indicate more drowsiness. In some embodiments, a longer blink duration may indicate more drowsiness. Some embodiments include biasing or compensating such inferences based on demographic information, or other relevant characteristics, of the individual. The demographic information can include one or more of ethnicity, age, or gender.

Other eye blink characteristics, or changes in such characteristics, such as, for example, changes in eye blink rates, can be related to drowsiness. For example, in certain cases, drowsiness can be determined based on eye blink rates increasing and the duration of eyelid closure increasing. Drowsiness or somnolence can include a mental state in which an individual has a strong desire to sleep. Drowsiness can include a mental state that can precede the individual falling into a sleep state. Drowsiness can be an indicator that an individual is unfit to operate machinery, operate a motor vehicle, or other tasks which require that the individual be alert. Drowsiness can be due to a variety of factors including lack of sleep, physical health, mental health, medical conditions, diet and/or nutritional characteristics, time of feeding, medications, illicit drugs, and other factors. While a range of treatments can be applied to drowsiness, the presence of drowsiness as a mental state of an individual can be used to make recommendations to the individual such as, for example, to not operate machinery or a motor vehicle, to not engage in complex tasks, to not engage in potentially hazardous activities, and so on.

Further, eye blink characteristics, or changes in such characteristics, such as, for example, changes in eye blink rates, can be related to fatigue. For example, in certain cases, eye blink rates can decrease as fatigue increases. Fatigue can include eye fatigue, where eye fatigue can result from long periods of time on a task (ToT), viewing a computer screen or television, operating a motor vehicle, and so on. Fatigue can include tiredness and can occur gradually. The effects of fatigue can generally be reversed by the individual getting rest or getting appropriate treatment for relevant medical conditions. Fatigue can be based on physical causes and mental causes. Physical causes of fatigue can result from strenuous physical activity and can result in the muscles of an individual being unable to operate at an optimal level of physical performance. Mental fatigue can result from intense mental activity and can cause, for example, a diminution of cognitive performance. Fatigue can result from hard, physical labor or long hours at the office, mental stress, overstimulation, under stimulation, active recreation, jet lag, ennui, mental health conditions, such as depression, disease, sleep deprivation, and the like. Fatigue may also result from chemical causes such as vitamin or mineral deficiencies and poisoning, or, for example, caloric deprivation or overconsumption. Fatigue can result from afflictions such as the common cold or influenza, and medical conditions such as anemia.

In embodiments consistent with the present disclosure, relevant eye blink information can include eye-blink rate, eye-blink duration, time between blinks, patterns of eye blinks, blink phase, blink amplitude, and/or other information related to one or more eye blinks by an individual, including, for example, changes in such characteristics over time. That is, embodiments consistent with the present disclosure can determine such eye blink information in an individual.

Once the eye blink information is determined, the eye blink information can be correlated with context, for example, the activity being performed by the user, demographic information about the user, such as the user's age and/or gender, the time of day or the time within the individual's sleep cycle, the brightness of the screen and/or the environment, or other contextual information. In some embodiments, the eye-blink information is compensated, or adjusted, based on the context; for example, one or more thresholds at which a mental state is ascertained based on eye blink information may be adjusted based on the context. The eye blink information can then be used to infer the mental state of the individual, which is correlated to context in some embodiments. The mental state can be used to, for example, modify an activity being performed by the individual, a game being played, a choice of advertisement to be displayed, a media presentation, or some other activity. In some embodiments, an output is rendered where such output displays the mental states and/or eye blink information, which can be correlated with the context. For example, an output may be rendered, where such output displays mental states and/or eye blink information over the course of a timeline of a media presentation.

In some embodiments, the presence or absence of fatigue in an individual is ascertained or evaluated by obtaining a blink measurement, which in embodiments, may include, for example, one or more of detecting the amplitude, frequency, opening time, phase, and/or pattern of one or more blinks. Evaluating fatigue may additionally comprise determining or measuring head nodding, such as, for example, with a gyroscope or accelerometer or other applicable sensor. In other embodiments, evaluating fatigue may additionally comprise evaluating or detecting or measuring eye movements, such as, for example, when the subject is performing smooth pursuit, saccadic and/or opticokinetic eye movement. In some embodiments, the method or device includes triggering eye movement with an inducer of smooth pursuit, saccades and/or opticokinetic eye movement. In some embodiments, a device with three electrodes (or, in some embodiments, no more than three electrodes), configured for positioning in a mono-ocular fashion, i.e., only on one side of a face, is provided. In other embodiments, a device with only two electrodes, configured for positioning in a mono-ocular fashion only on one side of a face, is provided.

### A. Device and System for Detecting Eye Blinks

To assess fatigue, a device with sensors to detect or derive electrical activity, e.g., corneo-retinal potential signals or other electrical signals related to an eyelid blink (i.e., an eye blink), and/or eye movement of a subject and circuitry (e.g., a circuit) operably coupled to the sensors is provided. Additional information regarding devices of the present invention, including embodiments of exemplary devices, are described in International Publication No. WO2021/141850 as well as in U.S. Provisional Patent Application Ser. No. 63/525,241, filed Jul. 6, 2023, in each case, incorporated by reference herein.

In an embodiment, the device comprises a unitary substrate including a first electrode, a second electrode, and circuitry operably coupled to the first electrode and the second electrode. In embodiments, the unitary substrate is dimensioned for unilateral placement on a subject's face to position the first electrode and the second electrode to detect electrical activity associated with an eye blink of the subject and/or electrical signals correlated with eye movement, such as horizontal and vertical eye movement. In embodiments, the device is a wearable device, such as a portable wearable device. For example, a device can be worn by a subject outside of a clinical setting, such as at home or at work. In some cases, a device is configured to be portable such that the user can easily move locations while the device is used substantially continuously.

In another embodiment, the device, e.g., the wearable device, for monitoring eye blinks and/or eye movement of a subject includes first and second sensors configured to sense characteristics of one or more eye blinks of the subject and/or eye movement of the subject, and circuitry operably coupled to the sensors and configured to detect characteristics of one or more eye blinks of the subject and/or horizontal and/or vertical eye movements based on signals from the first and second sensors. The terms sensor or sensing unit are used herein to refer to both electrodes and other sensors. The device, in another embodiment, includes a transmitter. In embodiments, the transmitter is configured to transmit signals sensed by the first and second sensors to remote circuitry configured to receive signals transmitted by the transmitter and to detect characteristics of one or more eye blinks of the subject and/or horizontal and/or vertical eye movement based on signals from the first and second sensors. In another embodiment, the device, e.g., the wearable device, includes first and second sensors configured to sense characteristics of one or more eye blinks of the subject and/or eye movement of a subject; and (i) circuitry operably coupled to the sensors and configured to detect characteristics of one or more eye blinks of the subject and/or horizontal and vertical eye movements based on signals from the sensors, and/or (ii) a transmitter configured to transmit data originating from the first and second sensors to remote circuitry configured to detect characteristics of one or more eye blinks of the subject and/or horizontal and vertical eye movements based on signals from the sensors. In these embodiments, each sensor may include a single electrode, two electrodes, three electrodes, four electrodes, or more.

The device may also comprise a storage component. In this embodiment, the circuitry and the storage component are configured to record eye blink data and, in some cases, head movement, position and/or orientation data onto the storage component. By record eye blink data and, in some cases, head movement, position and/or orientation data onto the storage component, it is meant electronically retain signals sensed by the first and second sensors and the third sensor or processed signals or information derived from signals sensed by the first and second sensors and the third sensor onto a persistent memory storage device such that the stored data can be accessed at a later time.

By storage component, it is meant an electronic component capable of having electronic data written onto it and read from it, such that data written thereon persists over time in a manner that it can be accessed and read at a later time. Typically, the storage components integrated into the device are commercially available. “off-the-shelf” components. For example, flash memory storage components are commercially available from Intel or Samsung or Toshiba.

The storage component may be removable. For example, the storage component may be a removable memory card such as an SD card or the like. By removeable, it is meant that the storage component may be configured such that it can be physically and electronically separated and removed from the circuitry of the device and later physically and electronically reintegrated into the device. The storage component might be separated from the device so that data on the storage device can be read and downloaded onto a remote computer system.

In certain embodiments, the device may be configured to record eye blink data and, in some cases, head movement, position and/or orientation data onto the storage component in near real time. By recording eye movement data and, in some cases, head movement, position and/or orientation data onto the storage component in near real time, it is meant that the device is configured to store signals sensed by the first and second sensors and the third sensor or signals or data derived from the signals sensed by the first and second sensors and the third sensor nearly in real time after signals are sensed by the first and second sensors and third sensor and received by the circuitry.

In some embodiments, the transmitter, discussed supru, may be a wireless transmitter. By wireless transmitter, it is meant that the transmitter receives an electronic signal and produces electromagnetic waves via an antenna corresponding to that signal that can be received by a receiver that is remote, meaning electronically and physically separate from the device. In some instances, the wireless transmitter may be a wireless network interface controller, meaning, for example, a device capable of connecting via radio waves to a radio-based computer network. Alternatively, the wireless transmitter may be a Bluetooth interface controller, meaning, for example, a device capable of connecting via the radio waves to a Bluetooth-enabled remote device. Typically, the transmitters integrated into the device are commercially available, “off-the-shelf” components. For example, wireless network interface controllers are commercially available from Intel or Nordic or Qualcomm, and Bluetooth interface controllers are commercially available from Motorola or Nordic or Qualcomm.

In some embodiments, the device may be configured to transmit eye blink data and head movement, position and/or orientation data via the transmitter in near real time. By transmitting eye blink data and head movement, position and/or orientation data via the transmitter in near real time, it is meant that the device is configured to transmit signals sensed by the first and second sensors and the third sensor or signals or data derived from the signals sensed by the first and second sensors and the third sensor nearly in real time after signals are sensed by the first and second sensors and third sensor and received by the circuitry.

As will be described in greater detail below, in embodiments, the wearable device functions as part of a system that comprises a wearable device and a software application on a mobile device. The software application may have an algorithm for analysis of data from the wearable device, and other features that are described infra.

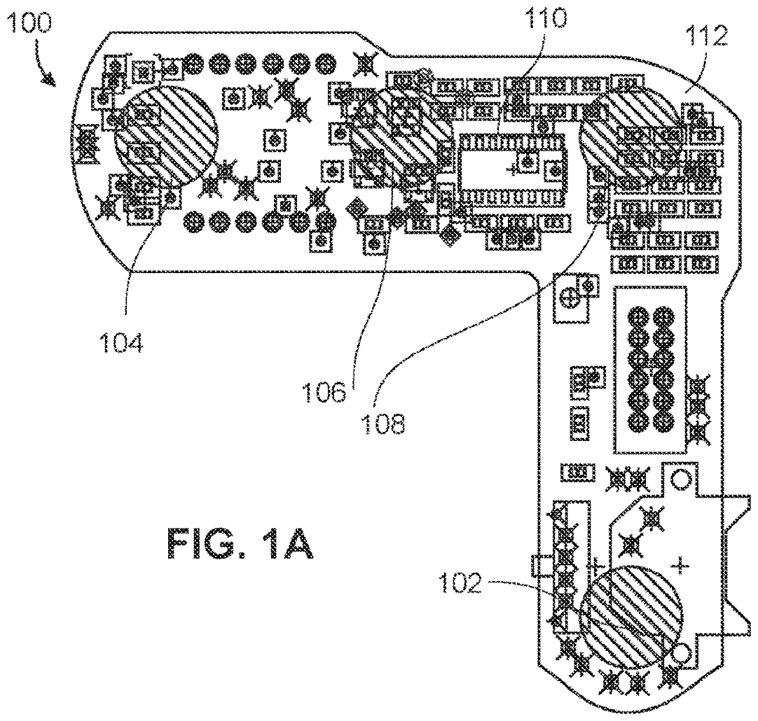

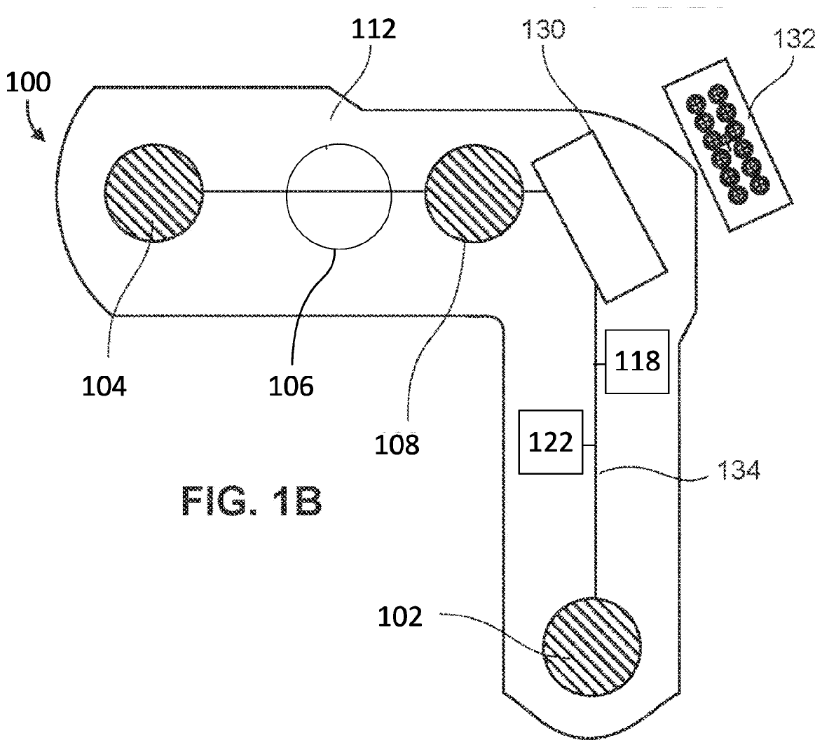

FIGS. 1A-1B illustrate back/front views, respectively, of a blink detector 100 including a first sensor 102 and a second sensor 104, according to some embodiments. The embodiment of blink detector 100 also includes additional optional sensors, 106, 108. Sensors 102, 104, 106, and 108 may, in each case, include a single electrode; however, it is contemplated that a sensor may also include two or more electrodes. It is also contemplated that sensors 102, 104, 106, and 108 be components other than an electrode; for example, in some embodiments, any one of sensors 102, 104, 106, and 108 may include an accelerometer, a gyroscope, a magnetometer, or an inertial mass unit, for example. Blink detector 100 also includes circuitry 110. Sensors 102, 104, 106 and, 108, and the circuitry 110 are integrated onto a single, unitary substrate 112. Substrate 112 may be a printed circuit board, although other embodiments are possible and are contemplated, on which sensors 102, 104, 106, and 108 are mounted, embedded, or affixed. In one embodiment, any one of sensors 102, 104, 106, or 108 are removably insertable onto or into substrate 112. A compartment 130 is positioned at least partially on the front side of substrate 112 and is dimensioned to receive, and optionally release, an electronic component 132. Electronic component 132 may include a printed circuit board with electronics, described below. Electronic component 132 may be insertable, and preferably removably insertable, into compartment 130. When inserted into compartment 130, electronic component 132 is in electronic communication with sensors 102, 104, 106, and 108 via other elements of the circuitry such as traces or leads or other applicable electrical connections 134, on substrate 112. In some embodiments, sensors 102, 104, 106, and 108, and the circuitry are mounted into or onto—that is, are held together by—substrate 112. Substrate 112 may have an angled shape with two legs forming a preselected angle between them (e.g., an “L” shape, although other geometries are possible). In this embodiment, the first 102 and second 104 sensors or electrodes are separated from each other on substrate 112.

In some embodiments, blink detector 100 also includes a communications module 118 (i.e., a transmitter) configured to interface with a mobile device to send and receive information, such as data, requests, responses, and commands. Communications module 118 can include, for example, a network interface circuit and/or radiofrequency (RF) hardware and software (e.g., Wi-Fi, Bluetooth, NFC, and the like). Accordingly, blink detector 100 may be paired with a user device, such as a mobile device, to receive software updates, and to transmit data, i.e., upload data, to the device or to a network.

Blink detector 100, or portions thereof, can be reusable or disposable. For example, substrate 112 with sensors 102, 104, 106, and 108 can be reusable. In some embodiments, substrate 112 can be disposable and have removably insertable sensors 102, 104, 106, and 108 and/or electronic components that are reusable by insertion into a fresh, unused substrate 112.

When blink detector 100 is disposed on a headset or helmet or other wearable aspect worn by a subject, the locations of sensors 102, 104, 106, and 108 on the face of the subject are defined, at least in part, by their positions on substrate 112, and the location of blink detector 100 within the headset or helmet or other wearable aspect. In some embodiments, blink detector 100 is configured for unilateral placement on the face for monocular placement of sensors 102, 104, 106, and 108 (e.g., in the proximity of one of the subject's eye).

The constituent components of blink detector 100 may be integrated onto a single, unitary substrate 112. In some embodiments, substrate 112 includes an underlying material or layer that acts as a mechanical base on which the constituent components of blink detector 100 may be mounted and fastened. Substrate 112 may be a substantially flat surface where each constituent component occupies an area on a flat surface. Substrate 112 may be a substantially planar member, where each constituent component occupies an area on or within the planar member. Substrate 112 may comprise any convenient material, such as a flexible material, such as a biocompatible material. e.g., a flexible polymer, such as silicone rubber.

In an embodiment, the device comprises a single wearable patch or in other embodiments, the device comprises more than one wearable patch. By comprising a single wearable patch, it is meant that the substrate on which the device is mounted is itself integrated into a single wearable patch. By comprising more than one wearable patches, it is meant that the components that comprise the device are integrated onto more than one wearable patch, such that when worn, each wearable patch is physically separated from each other and may be electrically connected via wires or may be in wireless communication with each other.

The single wearable patch may take any convenient form and may include any convenient material or materials. For example, the single wearable patch may include a flexible material, such as a cloth-based material, that can be shaped over parts of the face of the subject. The single wearable patch can also be formed from layers of the same or different materials or may be a laminate of the same or different materials. When the device is configured to comprise a single wearable patch, the wearable patch may be adhered to a facial location of the subject or may be attached to a facial location of the subject using non-adhesive material. In certain embodiments, the single wearable patch may be adhered to a facial location of the subject using a glue or a cement or a paste. In certain embodiments, the single wearable patch may be attached to a facial location of the subject not by using an adhesive but instead by using, for example, a tensioning method, such as an elastic band or an eye glass frame. In some embodiments, the single wearable patch may be flexible so as to be fitted to a facial location of the subject. By flexible so as to be fitted to a facial location, it is mean that the surface of the single wearable patch is not rigid or firm but instead may be positioned so as to follow the pattern of and be aligned with the non-flat contours of a subject's face. In some embodiments, the single wearable patch may be moldable so as to be form-fitted to a facial location of the subject. By moldable so as to be form-fitted to a facial location of the subject, it is meant that the surface of the single wearable patch can be manipulated and formed so as to follow the pattern of and be aligned with the non-flat contours of a subject's face and, further, when so formed, will retain such molded position. In some embodiments, the single wearable patch may be configured to be torn so as to be fitted to a facial location of the subject. By being configured to be torn so as to be fitted to a facial location of the subject, it is meant that the material of the wearable patch is configured so as to guide a user to tear a flat surface of the wearable patch such that the otherwise flat surface of the wearable patch may better accommodate being applied to a more substantially rounded or curved surface of a subject's face.

Sensors 102, 104, 106, and 108 may sense movement of one of the subject's eyelids by measuring electrical activity associated with activation of one or more eyelid muscles. Accordingly, the first 102 and second 104 sensors may sense the movement of the subject's eyelid by measuring the electrical activity of the subject's muscles, for example, the extraocular and facial muscles. Sensors 102, 104, 106, and 108 may each include one or more electrodes, or a single electrode. The electrodes may be surface electrodes, in which case they may be dry electrodes or wet electrodes, such as in each case commercially available electrodes. In certain embodiments, blink detector 100 may be configured to monitor the subject's eyelid movement based on signals from the first and second sensors continuously and/or in near real time.

The electrodes in sensors 102, 104, 106, and 108 may include any convenient electrode. By electrode, it is meant an electrical conductor used to make contact with a nonmetallic substance. In some instances, the electrodes are integrated into blink detector 100 such that one end of the electrode is in electrical contact with the subject, and the other end of the electrode is electrically connected to electric component 132. In some instances, the electrodes have a proximal and distal end, wherein the proximal end is electrically connected to electric component 132, and the distal end is in electrical contact with the subject when in use. Thus, an electrode may be used to conduct electrical signals generated by the subject and sensed by the electrode to electric component 132.

In certain embodiments, the electrodes are surface electrodes. By surface electrodes, it is meant electrodes that are applied to the outer surface of a subject to measure electrical activity of tissue proximal to the electrode. That is, surface electrodes are electrodes that do not penetrate the skin of a subject. Surface electrodes may be pre-gelled electrodes. Surface electrodes may be any convenient commercially available surface electrode, such as the Red Dot™ line of electrodes that are commercially available from 3M™ or Disposable Surface Electrodes that are commercially available from Covidien or ECG Pre-Gelled electrodes that are commercially available from Comepa. In certain embodiments, surface electrodes may include a backing material that is a cloth, a foam, a plastic tape, a plastic film, or any other convenient material. Surface electrodes may be applied to the surface of the subject using an adhesive. The strength of the adhesive may vary as desired. The adhesive may itself be conductive, i.e., electrically conductive. The surface electrodes may be designed to be conveniently repositioned on a subject as needed to improve functioning of the device.

In certain embodiments, the surface electrodes may be dry electrodes. By dry electrode, it is meant that the electrodes do not require the application of any gel or other fluid between the subject's skin and the distal surface of the electrode for the electrode to function in the device. In certain embodiments, the dry surface electrodes do not require any skin preparation, such as skin abrasion, in order to function when applied to the surface of a subject. In other embodiments, the dry surface electrodes do require skin preparation, such as skin abrasion, prior to bringing the device into contact with the subject, in order to facilitate or improve functioning of the device. When the electrodes are not dry electrodes, gel or other similar fluid is applied to the surface of the subject between the skin and the electrode in order to promote electrical connectivity between the subject and the surface electrode. In certain instances, dry electrodes promote long-term use of the electrodes and therefore long-term use of the device by alleviating the need to reapply gel or similar fluid as the gel or other fluid dries or evaporates away. In some cases, wet electrodes offer an advantage of acquiring a stable signal for longer periods of time.

In some embodiments, blink detector 100 includes groups of sensors, the groups of sensors 102, 104, 106, and 108 geometrically arranged in different configurations around the subject's face. In some instances, sensors 102, 104, 106, and 108 may be geometrically arranged symmetrically with respect to a vertical or horizonal axis through the pupil of the subject. In some instances, sensors 102, 104, 106, and 108 may be geometrically arranged asymmetrically with respect to a vertical or horizonal axis through the pupil of the subject. In some instances, one or more groups of sensors 102, 104, 106, and 108 may be geometrically arranged to isolate certain electrical activity, such as, for example, electrical activity associated with certain characteristics of eye blinks, or, in other cases, to isolate vertical eye movements of the subject, e.g., to isolate such movements from eye blinks from the subject. In some instances, sensors 102, 104, 106, and 108 may be geometrically arranged to isolate horizontal eye movements of the subject, e.g., to isolate such movements from eye blinks from the subject. In some instances, sensors 102, 104, 106, and 108 may be geometrically arranged to isolate torsional eye movements of the subject, e.g., to isolate such movements from eye blinks of the subject. In some instances, sensors 102, 104, 106, and 108 may be geometrically arranged to isolate vertical, horizontal, and torsional eye movements of the subject from eye blinks from the subject.

In some embodiments, signals from sensors 102, 104, 106, and 108 may be received by electronic component 132 simultaneously, quasi-simultaneously, or overlapping in time. That is, in some instances, signals from the different sensors 102, 104, 106, and 108 may be multiplexed prior to being received by the electronic component 132. In some instances, signals from sensors 102, 104, 106, and 108 may be multiplexed such that electronic component 132 receives signals from each sensor 102, 104, 106, and 108 in substantially equal durations of time. In other instances, signals from sensors 102, 104, 106, and 108 may be multiplexed such that electronic component 132 receives signals from one of sensors 102, 104, 106, and 108 for a greater duration of time than from another one of sensors 102, 104, 106, and 108.

Electronic component 132 may include an analog front end and a digital circuit. In embodiments, the analog front end receives signals from the sensors and may include a noise filtering circuit and an amplifier circuit. The digital circuit may include an analog to digital converter circuit configured to convert the analog signal output from the analog front end into a digital signal as well as a microcontroller. In some embodiments, the digital circuit may also include a digital signal processor to further process the signal output from the analog front end. In embodiments, the digital circuit may comprise any other convenient processor, such as, for example, a general-purpose or other specific-purpose processor or logic circuit (e.g., an application specific integrated circuit). In some cases, the device comprises a processor electrically coupled to first and second electrodes and is configured to provide a signal representative of the electric potential detected by the first and second electrodes.

In some embodiments, at least one of sensors 102, 104, 106, and 108 may be configured to detect head movement, position, and/or orientation based on signals from such sensor. In certain embodiments, any one of sensors 102, 104, 106, and 108 may include a photosensor configured to detect ambient light based on signals from the photosensor. In other embodiments, blink detector 100 further includes a storage component 122 communicatively coupled with electrical component 132. Storage component 122 may be configured to record eye blink data, head movement, position, orientation and/or other data, in each case provided by sensors 102, 104, 106, and 108. In some embodiments, communications module 118 is configured to transmit eye blink data and head movement, position, orientation and/or other data, e.g., to transmit such data to a remote device, such as a mobile device, e.g., a smartphone or tablet or laptop computer or the like.

Electronic component 132 may include an electronic circuit, in which electrical signals are conveyed via electrical conductors and the voltage and/or current of the electrical signals may be manipulated by electronic components, such as, for example, resistors or capacitors or voltage sources or current sources and the like. Electronic component 132 may further include semiconductor devices, such as transistors or integrated circuits or processors and the like. The electronic components comprising the circuitry may consist of both analog and digital electronic components.

In some embodiments, analog components of electronic component 132 may include one or more amplifiers, such as, for example, operational amplifiers, and one or more analog filters, such as, for example, low-pass filters, high-pass filters, or band-pass filters. In some embodiments, one or more amplifiers is used in electronic component 132 to amplify electronic signals originating from sensors 102, 104, 106, and 108. In particular, one or more amplifiers and analog filters may be used in electronic component 132 to amplify and filter aspects of the signals received from sensors 102, 104, 106, and 108 that are associated with eye blinks of the subject. In addition, one or more amplifiers and analog filters may be used in the circuit to condition the signals received from the first and second electrodes such that they can be further processed in electronic component 132.

In some embodiments, electronic component 132 may include an analog to digital converter, a microcontroller, and a digital signal processor or any other convenient processor, such as, for example, a general-purpose processor or other specific-purpose processor or logic circuit (e.g., an application specific integrated circuit). By analog to digital converter, it is meant an electronic circuit used to convert electrical signals from an analog format or encoding to a digital format or encoding. In certain embodiments, an analog to digital converter may be used to convert analog signals that have already been processed by the analog electronic components of the circuit into digital signals, such that the resulting digital signals can be further processed by electronic component 132. By microcontroller, it is meant an electronic circuit that includes one or more processors, memory, and one or more input/output interfaces. In certain embodiments, the microcontroller may be programmed to further process the digital signal corresponding to the signal measured by the first and second sensors or electrodes as well as the digital signal corresponding to the signal measured by a third sensor, such as an accelerometer, and the digital signal corresponding to the signal measured by a photosensor. The microcontroller may also be programmed to facilitate transmitting a digital signal via communications module 118 or to facilitate storing a digital signal onto storage component 122. The microcontroller may also be programmed to fetch analog to digital convert (ADC) converted data, to schedule data communication, and to facilitate local signal processing. By digital signal processor, it is meant a special purpose microprocessor that is optimized for performing signal processing operations, such as measuring digital signals, comparing digital signals against reference waveforms, filtering digital signals, or compressing digital signals and the like. In certain embodiments, the digital signal processor may be used to: scale and bias raw sensor measurements; identify characteristic waveforms by comparing measured waveforms against a reference waveform; identify specific characteristics of measured waveforms; or compress the digital representation of the waveform prior to transmitting or storing the waveform. For example, when the digital signal processor is used to identify characteristic waveforms of the digital signal corresponding to the signal measured by the first and second sensors, the digital signal processor may compare the measured signal against reference waveforms corresponding to an eye blink, or reference waveforms corresponding to other eye movements, characteristic eye movements, such as for example, neutral gaze, leftward gaze, rightward gaze, upward gaze and downward gaze, or reference waveforms corresponding to characteristic eye movements associated with a nystagmus event, or reference waveforms corresponding to characteristic eye movements associated with horizontal nystagmus events, vertical nystagmus events and torsional nystagmus events, or reference waveforms corresponding to characteristic eye movements of nystagmus events associated with benign paroxysmal positioning vertigo, or reference waveforms corresponding to characteristic eye movements of nystagmus events associated with Menière's disease, or reference waveforms corresponding to characteristic eye movements of nystagmus events associated with vestibular neuritis.

In some embodiments, the electronic component 132 may be provided in the form of integrated circuits. For example, one or more of the electronic components described above may be provided on an application specific integrated circuit (ASIC). Alternatively, in some embodiments, electronic component 132 may include a configurable integrated circuit. For example, electronic component 132 may include a field programmable gate array that has been configured to implement identical functionality.

In some embodiments, the electronic components that make up the circuitry are commercially available, “off-the-shelf” components. For example, integrated circuits comprising operational amplifiers, analog filters, analog to digital converters, microcontrollers and digital signal processors are commercially available from Texas Instruments or Analog Devices and Marvell. Field programmable gate arrays that can be configured to implement one or more of the electronic components described above are commercially available from Xilinx or Intel and Altera.

The algorithm that processes a signal from the circuitry is able to detect eye blinks based on signals from sensors 102, 104, 106, and/or 108. In some embodiments, the algorithm may be configured to detect additional eye movements based on signals from sensors 102, 104, 106, and/or 108, such as torsional eye movements, one or more specific patterns of eye movement sensor data including one or more characteristic eye movements, including, for example, recognizing neutral gaze, leftward gaze, rightward gaze, upward gaze and downward gaze, or, a nystagmus event, including horizontal nystagmus events, vertical nystagmus events and torsional nystagmus events, or nystagmus events associated with benign paroxysmal positioning vertigo, or nystagmus events associated with Menière's disease, or nystagmus events associated with vestibular neuritis. In certain embodiments, when sensors 102, 104, 106, and/or 108 are positioned proximal to one eye of a human subject, the algorithm may be configured to detect eye blinks by first receiving electrical signals in an analog format that is correlated with eye blinks. By a signal correlated with eye blinks, it is meant, for example, a signal that represents an electrical signal that represents the electrical activity of muscles. By electrical activity of muscles, it is meant that sensors 102, 104, 106, and/or 108 are configured to measure electrical activity of extraocular and facial muscles associated with eye blinks and/or eye movement. Upon receiving such analog electrical signals, electronic component 132 may be configured to selectively amplify the signal using an analog amplifier, as described above, to particularly amplify, for example, the part of the signal that includes the electrical activity of extraocular and facial muscles associated with eye blinks or eye movement. In one embodiment, a signal that includes a dipole fluctuation between cornea and retina (indicative of vertical or horizontal eye movement) is removed or subtracted from the signal collected from the device.

Upon amplifying the signal, electronic component 132 may be configured to filter the amplified signal using an analog filter, as described above, to exclude and remove parts of the analog electronic signal, for example, in certain cases, to remove parts of the analog electronic signal that do not correspond to, for example, measurements of the difference in electrical potential between a cornea and a retina of the subject. Upon amplifying and filtering the analog electronic signal, electronic component 132 may convert the analog signal into a digital signal using an analog to digital converter, as described above. Upon converting the signal into a digital signal, electronic component 132 may be configured to measure aspects of the signal using a digital signal processor, as described above, to identify characteristics of the digital signal that are associated with an eye blink. In certain embodiments, the digital signal processor may be configured to identify additional characteristics of the digital signal that are associated with eye blinks (e.g., blink speed, blink duration, blink amplitude or the like) or eye movements, such as torsional eye movements, one or more specific patterns of eye movement sensor data including one or more characteristic eye movements, including, for example, recognizing neutral gaze, leftward gaze, rightward gaze, upward gaze and downward gaze, or, a nystagmus event, including horizontal nystagmus events, vertical nystagmus events and torsional nystagmus events, or nystagmus events associated with benign paroxysmal positioning vertigo, or nystagmus events associated with Menière's disease, or nystagmus events associated with vestibular neuritis.

As mentioned above, the device may include a third sensor configured to sense head movement, position and/or orientation of the subject. By sensing head movement, position and/or orientation of the subject, it is meant that the device is configured to detect when the head of a subject is translated back and forth, up or down or side to side in space, as well as when the head is rotated from side to side or back and forth or up and down, or combinations thereof.

In some embodiments, the third sensor may be an accelerometer. By accelerometer, it is meant a component that measures the acceleration of a body, such as acceleration on three dimensions in space. The accelerometer may comprise a mechanical-electronic device or a microelectromechanical system (MEMS). For example, an accelerometer may utilize the piezoelectric effect to measure acceleration. Typically, the accelerometers integrated into the device are commercially available, “off-the-shelf” components. For example, integrated circuits comprising accelerometers are commercially available from Analog Devices or Texas Instruments or Marvell.

Alternatively, the third sensor may be a gyroscope. By gyroscope, it is meant a component that measures the changes in the position or rotation of a body, such as changes in the orientation of the body in three dimensions in space. The gyroscope may comprise a mechanical-electronic device or a microelectromechanical system (MEMS). For example, a gyroscope may be a vibrating structure gyroscope designed to utilize the piezoelectric effect to react to Coriolis force and thereby measure rotation of the sensor. Typically, the gyroscopes integrated into the device are commercially available, “off-the-shelf” components. For example, integrated circuits comprising gyroscopes are commercially available from Analog Devices or Texas Instruments or Marvell. The third sensor can also be a magnetometer or an inertial measurement unit.

In some embodiments, the device may be configured to monitor the subject's head movement, position and/or orientation based on signals from the third sensor continuously and/or in near real time. By monitoring the subject's head movement, position and/or orientation continuously, it is meant that the device may be configured to monitor the subject's head movement, position and/or orientation based on substantially every signal sensed by the third sensor. By monitoring the subject's head movement, position and/or orientation in near real time, it is meant that the device is configured to analyze and evaluate the subject's head movement, position and/or orientation nearly in real time after the signals are sensed by the third sensor and received by the circuitry.

Certain embodiments of the device may include a photosensor configured to sense ambient light in the vicinity of the subject. By ambient light in the vicinity of the subject, it is meant the intensity of light that is proximal to the subject wearing a device configured to include a photosensor. In some cases, ambient light also includes other characteristics of light, such as changes in light intensity or changes in wavelength characteristics of light proximal to the subject.

By photosensor, it is meant an electronic component capable of converting light into electronic current, such as a photodiode, such that the resulting electronic current can be measured by the circuitry of the device. Typically, the photosensors integrated into the device are commercially available, “off-the-shelf” components. For example, integrated circuits comprising photosensors are commercially available from Texas Instruments or Analog Devices or Marvell.

In some embodiments, the device may be configured to monitor ambient light in the vicinity of the subject based on signals from the photosensor substantially in real time. By monitoring the ambient light in the vicinity of the subject in near real time, it is meant that the device is configured to analyze and evaluate characteristics of ambient light in the vicinity of the subject nearly in real time after signals are sensed by the photosensor.

Where desired, the devices described herein may include any one of a variety of different types of power sources that provide operating power to the device components, e.g., as described above, in some manner. The nature of the power source may vary and may or may not include power management circuitry. In some instances, the power source may include a battery. When present, the battery may be a onetime use battery or a rechargeable battery. For rechargeable batteries, the battery may be recharged using any convenient protocol, including, but not limited to, wireless charging protocols such as inductive charging. In some applications, the device may have a battery life ranging from 0.1 hours to 120 days, from 14-30 days, from 8 hours to 30 days, from 8 hours to 12 days, from 12 hours to 24 hours, from 0.5 to 10 hours.

Additionally, in some embodiments, the device may be waterproof. By waterproof, it is meant that the device is substantially resistant to water. That is, the device will continue to function correctly and consistently notwithstanding the presence of water proximal or on the device. For example, the device may be configured to function when worn by a subject outside in the rain or in a high humidity environment. In certain embodiments, the device may be configured to be waterproof by encasing the device in a housing, such as a plastic housing that itself is substantially waterproof.

In an alternative embodiment of the device, the device comprises first and second sensors configured to sense eye blink events of a subject; and a transmitter configured to transmit signals sensed by the first and second sensors to remote circuitry configured to receive signals transmitted by the transmitter and to detect eye blink events based on signals from the first and second sensors. By first and second sensors configured to sense eye blink events of a subject, it is meant first and second sensors as described in detail herein. By transmitter configured to transmit signals sensed by the first and second sensors, it is meant a transmitter as described in detail herein. By remote circuitry, it is meant any convenient circuitry that may be configured to receive and process signals measured from the first and second sensors. By remote, it is meant a location apart from the components that are interacting with the subject during use. For example, a remote location could be another location, e.g., different part of a room, different room, etc., in the same vicinity of the subject, another location in a vicinity different from the subject, e.g., separate portion of the same building, etc., another location in a different city, state, another location in a different country, etc. As such, when one item is indicated as being “remote” from another, what is meant is that the two items are at least in different locations, not together, e.g., are one to five or more feet apart, such as ten or more feet apart, including 25 or more feet apart.

Unlike the embodiments of the devices described above, this alternative embodiment of the device may not comprise circuitry. Instead, as described above, the device comprises a transmitter that transmits signals to remote circuitry configured to receive signals from the transmitter and detect eye blink events based on signals received from the transmitter. In some embodiments, the remote circuitry may be configured to detect eye blink characteristics, such as those described herein, including for example, blink frequency, blink velocity, blink amplitude, blink clusters.

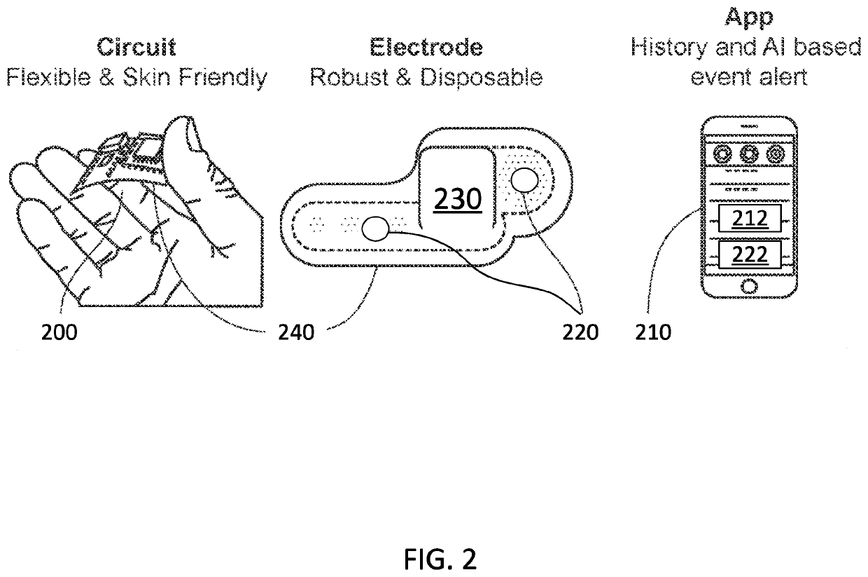

FIG. 2 illustrates a blink detector system 200 including a wearable device 240 and a software application installable onto or installed on a mobile device 210, according to one embodiment. Wearable device 240 may include one or more sensors 220, and circuitry 230. The system also includes a software application downloadable to, or as illustrated, downloaded onto a mobile device 210. Mobile device 210 may be any convenient mobile device such as a tablet or a smart phone.

Sensors 220 are configured to sense electrical signal associated with the subject's eye blinks. In some embodiments, sensors 220 are surface electrodes. Circuitry 230 is insertable, preferably removably insertable, into device 240, for operable connection to sensors 220. Mobile device 210 includes a processor 212 operably coupled to a memory 222 that includes instructions stored thereon for interfacing with the wearable device 210 as well as an additional storage component. Processor 212, memory 222, and instructions stored thereon of the mobile device 210 of the system 200 may be configured to apply an algorithm to the data detected by sensors 220. In some embodiments, the algorithm applied to the data may be an algorithm for recognizing certain eye blink events or for recognizing characteristic eye movements. For example, processor 212, memory 222, and instructions stored thereon may be configured to apply an algorithm to recognize eye blinks from a subject. The algorithm applied to the data detected by sensors 220 may comprise a machine learning algorithm, i.e., a machine learning model.

In some embodiments, a machine learning algorithm or a machine learning model may comprise one or more of: a statistical model, a linear model, a computational model, a tree-based model, a convolutional neural network, an artificial neural network, a deep learning network, a language model, such as a large language model (LLM), transformer-based models, models trained using at least in part, attention-based training mechanisms, as each such model and technique for training and applying it is known in the art. In embodiments, a machine learning model may comprise linear and tree-based baseline models, autoML-based boosted and ensemble models, deep learning models and large language models (LLMs). In certain cases, training the machine learning model comprises one or more of: an unsupervised learning technique, a semi-supervised learning technique, a supervised learning technique, parallel training techniques or attention-based training techniques, as each such technique is known in the art.

In embodiments, training a machine learning model to recognize eye blinks from a subject comprises utilizing one or more of stratified sampling, held out test set, cross validation, hyper parameter tuning or random grid search. In some cases, evaluating the accuracy of recognizing eye blinks from a subject by the model comprises using any available technique for assessing the accuracy of the model, such as, for example, using one or more of held out test sets and cross validation evaluations. In other cases, further training the machine learning model using electrical signal data and corresponding eye blink data comprises training the machine learning model using the plurality of electrical signal data and corresponding eye blink data in their entirety. For example, Long Short Term Models (LSTMs) are a type of recurrent neural network that have been shown to robustly classify time series data such as wearable ECG device data for heart disease. By applying LSTMs to signal data collected according to the present invention, fatigue or drowsiness detection may be determined or assessed with greater accuracy.

The first and second sensors in wearable device 240 are shown to each be a single electrode. Each such electrode may be attached to a substrate using a snap type fastener. Such fasteners removably engage with a member on the skin contact surface of device 240. Device 240 may be affixed to the skin of a subject by the electrodes that are secured, such as by an adhesive or by tape, to the skin, and by the fastening member on the device that removably engages with a mating member on an electrode. Device 240 may comprise an adhesive portion on the skin contact side in the region of electronic component 230, if there is no adhesive-backed third sensor electrode in this region of the device. Device 240 may also comprise fiducials or alignment markings to guide positioning of the device on the face of a user. For example, the alignment markings might indicate alignment with an eye or aspects thereof, such as the pupil. The alignment marking(s) may take the form of a removable layer that is peeled from the external surface of the device after it is affixed to the face.

In alternative embodiments, eye blink events of a subject may be detected by measuring electrical activity of the subject's eye at first and second locations by a system comprising a device with first and second sensors configured to sense eye blink events of the subject and a transmitter configured to transmit signals sensed by the first and second sensors to remote processing software, such as an algorithm, configured to detect eye blink events based on signals from the first and second sensors, and a mobile device. That is, the circuitry and/or the algorithm used to process signals from the first and second sensors may be remote from the sensors. In other instances, the movement of the subject's head may be detected by sensing the acceleration of the subject's head at a third location by the foregoing system wherein the constituent device further comprises a third sensor configured to sense head movement, position and/or orientation of the subject and wherein the transmitter is further configured to transmit signals sensed by the third sensor to the remote circuitry that is further configured to detect head movement, position and/or orientation based on signals from the third sensor. In still other instances, ambient light may be detected by sensing ambient light by the foregoing system wherein the constituent device further comprises a photosensor configured to sense ambient light and wherein the transmitter is further configured to transmit signals sensed by photosensor to the remote circuitry that is further configured to detect ambient light based on signals from the photosensor.

From the embodiments in FIGS. 1A-1B and FIG. 2, a variety of device configurations can be appreciated. In one embodiment, the wearable device comprises a reusable, non-disposable unitary substrate comprising the circuitry. First and second sensors or electrodes are removably attachable to the substrate, and may be disposed after use. The reusable, unitary substrate is attached to a fresh pair of unused electrodes or sensors for each subsequent use. In another embodiment, the substrate and electrodes of the device are disposable, and a majority of the circuitry is removable from the substrate for reuse. An example of this embodiment is depicted in FIG. 1B, where an electronic component of the circuitry is removable from the substrate. Devices that are entirely disposable or entirely reusable are also contemplated. In another configuration, the wearable device is comprised of a first sensor and a second sensor and an electronic component, where the electronic component is on a substrate separate from the substrate(s) on which the first sensor and second sensor are affixed. Circuitry between the separate substrates and sensor operably connects the sensor with the electronic component. Also contemplated is a device as described and comprising a skin adhesive layer and additional materials to assemble separate components together to form the device and affix it to the skin.

Accordingly, in embodiments, a device for adhesion to skin of a subject is provided. The device comprises a unitary substrate with a first arm and a second arm that join at a connection point, a first electrode positioned on the first arm and a second electrode positioned on the second arm. The device also comprises an electronic component, such as a data collection circuit, removably affixed at approximately the connection point. The first and second electrodes are integral with a bottom surface of each of the first arm and the second arm, respectively, and electrically connected to the electronic component when affixed to the substrate. The device may also comprise an adhesive on a bottom surface of each arm for contact with skin, and a flexible overlay covering the external, outward surfaces of the electrodes and the electronic component. The arms of the device may be flexible or rigid, depending on the materials from which the substrate is formed. In another embodiment, a device for adhesion to skin of a subject comprises a unitary substrate, which can be a layered substrate, with a first arm and a second arm and a data collection member or electronic component. First and second electrodes are removably affixed at separate individual connection points to the substrate, where the connection points are on a skin contact surface of each of the first arm and the second arm. The electronic component inserted onto or into the substrate is in electrical connection with the first and second electrodes. An adhesive may be present on the skin contact surface of the electrodes, and a flexible overlay material may cover the external, outward surfaces of the electrodes and the electronic component.

As described above, in embodiments, the mobile device is operably coupled with the wearable device such that data originating from the first and second sensors are accessible to the mobile device. For example, the mobile device and the wearable device may be operably coupled via a wired or wireless connection. By “mobile” is meant that the mobile device can be moved by the subject during use. For example, a mobile device could be carried in the subject's hand or the subject's pocket while the system is in use. Alternatively, the mobile device could be held by someone other than the subject during use, such as a health care provider or control room attendant. The mobile device includes a processor that is operably coupled to a memory that includes instructions stored thereon for interfacing with the device as well as an additional storage component. By operable coupling between the device and the mobile device, it is meant that the device and the mobile device are logically connected such that data originating from the first and second sensors are accessible to the mobile device. Any convenient protocol may be implemented to connect the device and the mobile device. For example, in certain embodiments, a wire or series of wires, i.e., a bus, may operably connect the device and the mobile device. Alternatively, a wireless connection, including a Bluetooth connection, may operably connect the device and the mobile device.

In embodiments, the mobile device may be any convenient mobile device. While the nature of the mobile device may vary, e.g., as described herein, in some instances the mobile device is a tablet or a smart phone. The mobile device may be a commercially available, “off-the-shelf” mobile device. For example, the mobile device could be, for example, an Apple iPhone or a Samsung Galaxy phone.

In some embodiments, the mobile device, such as mobile device 210, may further comprise a display. For example, the mobile device may include a digital readout, screen, monitor, etc. When the mobile device further comprises a display, the processor, the memory and the instructions stored thereon may be configured to display a graphical representation of data onto the display, in a graphical user interface (GUI) etc. For example, in different embodiments of the system, originating from the first and second sensors onto the display on the mobile device, the processor, the memory and the instructions stored thereon are configured to display a graphical representation of the data originating from the first and second sensors onto the display, or to display a graphical representation of the data originating from the first, second and third sensors onto the display, or to display a graphical representation of the data originating from the first, second and third sensors and the photosensor onto the display. The system may be configured to display a graphical representation of the data onto the display in near real time.

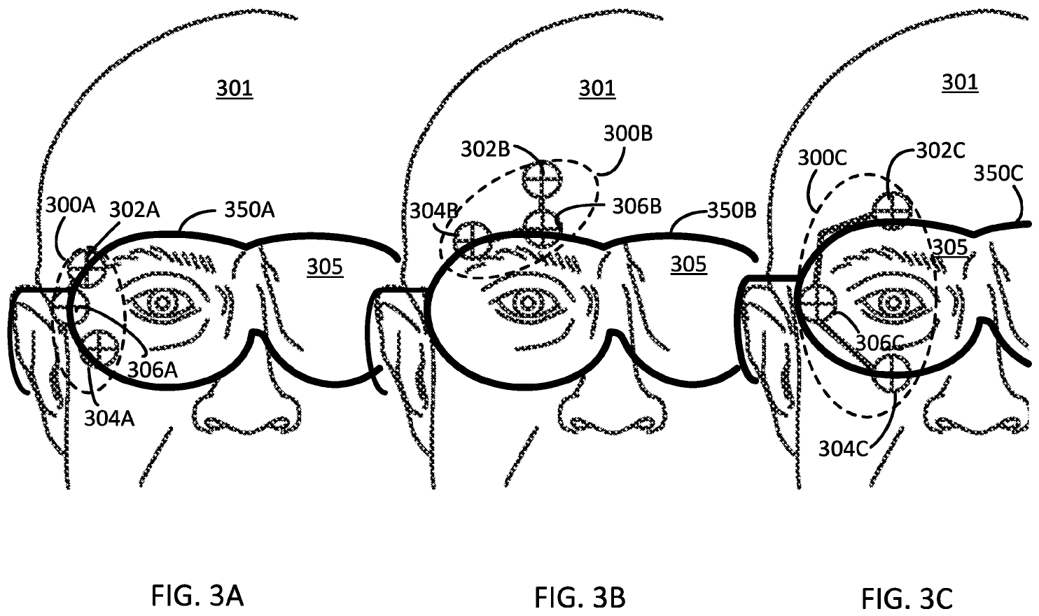

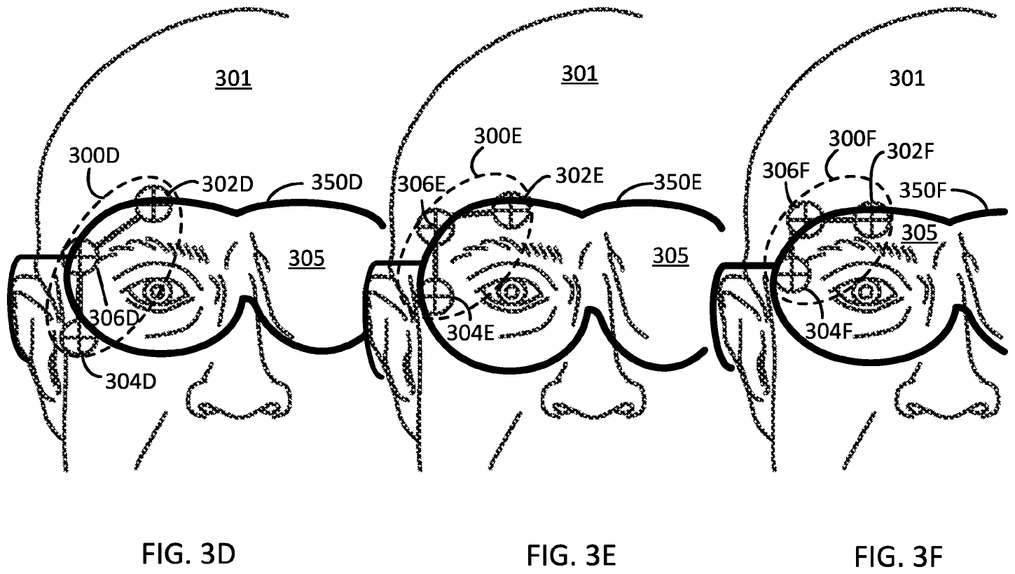

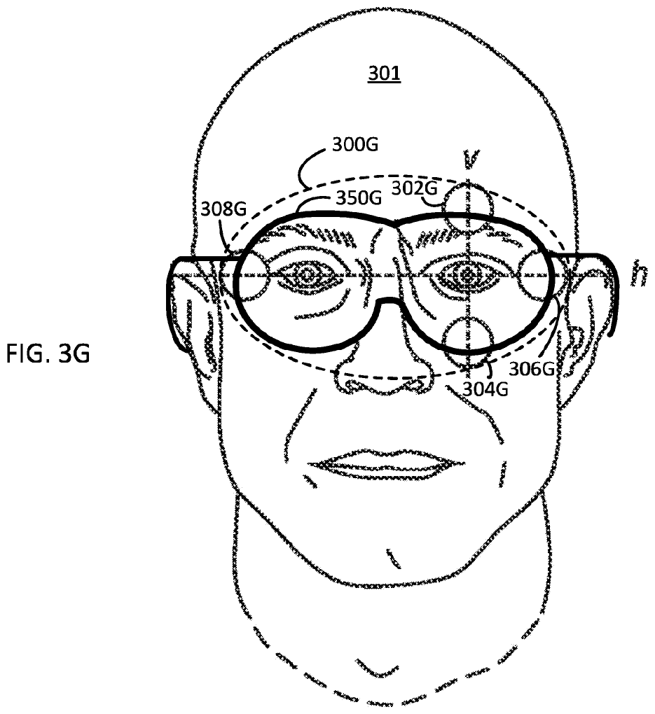

FIGS. 3A-3G illustrate goggles 350A, 350B, 350C, 350D, 350E, 350F, and 350G (hereinafter, collectively referred to as “goggles 350”), including blink detectors 300A, 300B, 300C, 300D, 300E, 300F, and 300G (hereinafter, collectively referred to as “blink detectors 300”), respectively, positioned on a user 301, according to some embodiments. Blink detectors may include sensors 302A. 302B, 302C, 302D, 302E, 302F and 302G (hereinafter, collectively referred to as “sensors 302”). Sensors 304A, 304B, 304C, 304D, 304E, 304F and 304G will be collectively referred to as “sensors 304.” Sensors 306A, 306B, 306C, 306D, 306E, 306F and 306G will be collectively referred to as “sensors 306.” Sensor 308G will also be referred to as “sensor 308.” Sensors 302, 304, 306 and 308 may comprises those sensors described above in reference to sensors 102, 104, 106 and 108.

Blink detector 300G includes binocular sensors 302G, 304G, and 306G (on the left eye), and 308G on the right eye. Because monocular corneo-retinal potential is lower than binocular corneo-retinal potential, blink detector 300 includes circuitry to amplify the corneo-retinal potential signal and to improve signal to noise ratio by incorporating sensor 308G on the right eye. The circuitry permits use of the signal from sensor 308G as a shared reference electrode, which allows sensors 302G, 304G, 306G, and 308G to not be perfectly aligned in the horizontal and vertical planes (h and v, respectively). In some embodiments, blink detector 300 includes an algorithm to decouple horizontal and vertical eye movement from signals provided by sensors 302G, 304G, 306G, and 308G, using binocular corneo-retinal potential. In other embodiments, sensors 302G, 304G, 306G, and 308G detect electrical activity from extraocular or facial muscles associated with a single eye.

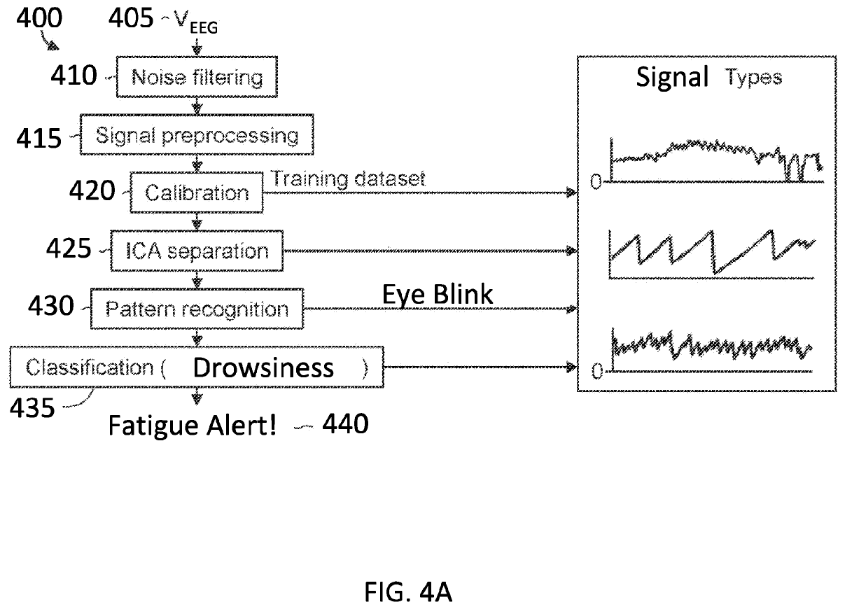

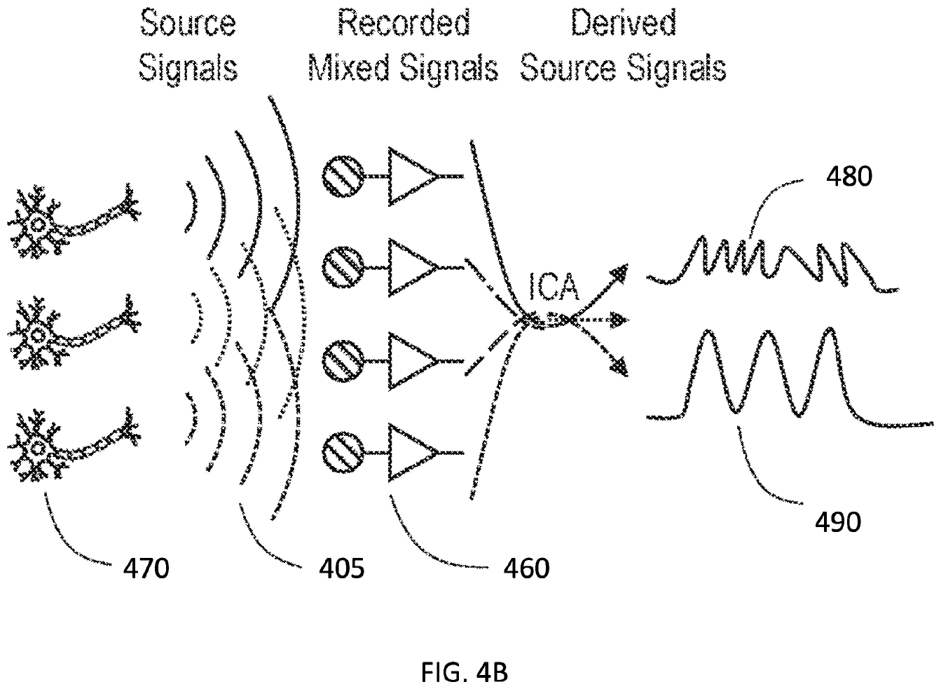

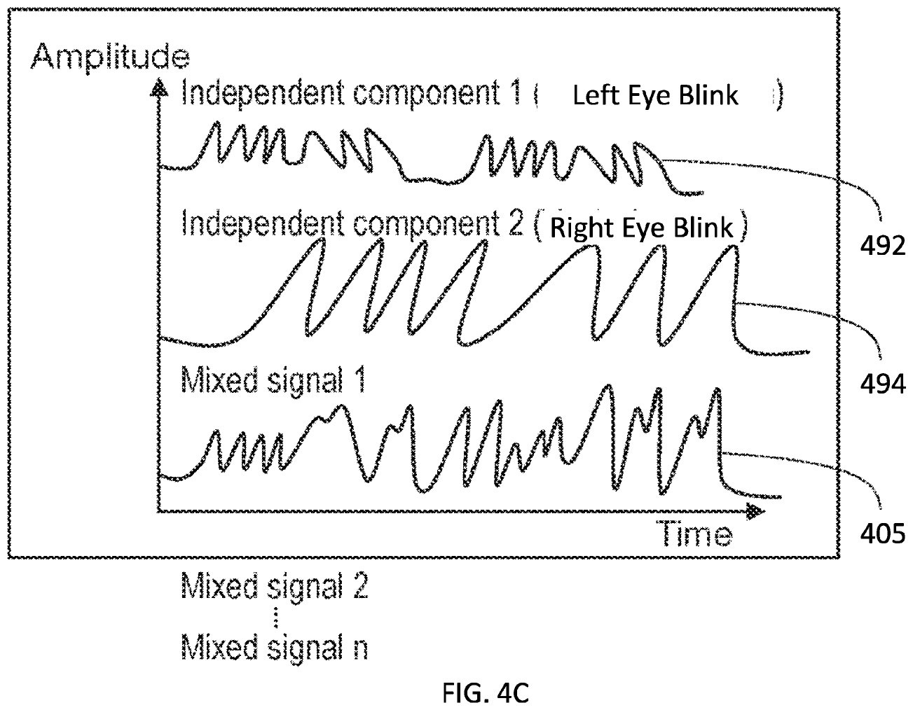

FIGS. 4A-4C illustrate a processing algorithm 400 and some exemplary signals 480, 490, 492, 494, and 405 obtained from a blink detector and used for assessing fatigue of a subject, i.e., a fatigue factor of a subject, according to some embodiments. The blink detector may include one or more sensors as disclosed herein (cf., sensors 102, 104, 106, 108, 302, 304, 306, and 308).

FIG. 4A shows the steps of an illustrative algorithm 400 as they are applied to an electroencephalogram (EEG) signal 405 originated from the sensors, according to one embodiment. In step 410, a noise filter is applied to signal 405 to filter out noise. For example, a 60 Hz noise filter can be applied to the signal to filter out 60 Hz noise. Step 410 may include applying a digital bandpass filter to filter out signals not between 0.1 to 100 Hz to avoid unwanted high frequency coupling. By way of example, when the desired eye blink or eye movement signal is close to 60 Hz, a 60 Hz digital notch filter may be applied to filter out environmental noise. In some embodiments, after filters are applied to the signal that originated from the sensors, the signal may be referred to as “cleaned.”

Step 415 includes preprocessing signal 405. Accordingly, step 415 may include scaling and/or biasing signal 405. For example, signal 405 may be biased to 0, to facilitate later processing. In some embodiments, a DC electrode offset cancellation may be desirable for sensing signals in a biological context (e.g., for signals below 100 Hz). Accordingly, in some instances, step 415 may include applying a DC electrode offset cancellation to signal 405.

Step 420 includes calibrating parameters in the algorithm as follows. In some embodiments, step 420 includes obtaining calibration data by following instructions displayed on a mobile device, on which the algorithm is downloaded or stored, with the subject. Such calibration data, obtained as the user follows the instructions displayed on the mobile device, is saved and labeled. The calibration data may be used for later training of more specialized algorithms, including machine learning or artificial intelligence algorithms, capable of classifying specific eye blink events or specific characteristics of eye blinks or specific types of eye movements. The calibration data may be used to condition, calibrate, or train one or more independent signal extraction algorithms, for example but not limited to, general independent signal extraction algorithms and independent component analysis (ICA) algorithms to conduct independent component analysis separation. In some instances, ICA separation is conducted using a machine learning algorithm. ICA separation may be used to “unmix” the distinct EEG signals associated with eye blinks and motion (e.g., a blink from the left eye, a blink from the right eye, or a simultaneous or quasi-simultaneous blink of both eyes) using signal 405.

Step 425 includes separating independent components of signal 405. In some embodiments, step 425 includes performing ICA separation using the calibration data on signal 405 to isolate distinct motions, and blinks, of the subject's eye. In some embodiments, step 425 may include the following mathematical steps: The observed EEG signal 405 from sensors in the eye blink detector may be written as:

$$x = As$$

Where, in the above, the vector x represents the recorded signals from the sensors; the vector s represents source signals (e.g., distinct signals corresponding to, for example, aspects of eye blink events, or vertical and horizontal components of eye motion, or left and right eye blinks), and the matrix A is a “mixing” matrix. To reconstruct the source signals, ICA separation may be applied. ICA is an algorithm for quickly finding the inverse of the matrix A. The inverse of matrix A is called the “unmixing” matrix and is commonly denoted as W. That is:

$$W = {Λ}^{- 1}$$

Therefore, it follows that:

$$s = W x$$

Thus, once the “unmixing” matrix, W, is computed, reconstructing the source signal, s (i.e., the distinct signals corresponding to, for example, aspects of eye blink events, or vertical and horizontal components of eye motion, or left and right eye blinks) is a matter of matrix multiplication between the “unmixing” matrix, W, and the recorded signals, x.

The ICA algorithm may be implemented in hardware or software in order to be applied to signals detected by sensors, as described herein. With respect to software implementations, any convenient software implementation may be applied. For example, open-source implementations of the ICA algorithm, such as Python's scikit-learn, may be applied as convenient.

Step 430 includes applying a pattern recognition to signal 405, wherein patterns of different eye motion and eye blink events may be identified.

Step 435 may include classifying one or more eye-related waveforms from EEG signal 405. For example, in some cases, step 435 may include classifying patterns of eye blinks as those associated with fatigue or drowsiness. In some embodiments, step 435 includes measuring an eye blink frequency, frequency pattern, blink duration, blink phase, an eye blink amplitude (e.g., how large is the subject's eyelid travel from maximum opening to minimum opening), a blink velocity (e.g., how fast the subject's eyelids are closed), identifying a blink “burst” (defined as multiple blinks in less than a pre-selected period of time) or changes in any such characteristics of a detected eye blink or eye blinks. Consequently, the system may issue a fatigue alert 440, when an assessment is made that the subject suffers from fatigue, drowsiness, exhaustion, and/or other physical/psychological stress.

FIG. 4B illustrates signal 405 measured by sensors 460 in a blink detector, according to an embodiment of the invention. Electrical activity in the underlying tissue 470 (e.g., including neurons coupled to muscles in the eye and eyelids) generates a complex EEG waveform that includes a linear mixing of two signals 480 and 490. Specifically, the signal measured by sensors 460 is a linear combination of a signal corresponding to a left eye blink or a right eye blink of the subject, sometimes even non-overlapping in time. ICA separation step 425 isolates signal 480 corresponding to a left eye blink and a signal 490 corresponding to a right eye blink. In some embodiments, at least one of signals 480 or 490 may correspond to another type of eye movement, such as a saccade or a nystagmus movement.

FIG. 4C illustrates how EEG signal 405 measured by the sensors in the eye blink detector is a linear mixing of two signals: a first independent component 492 corresponding to, for example, a left eye blink and a second independent component 494 corresponding to, for example, a non-overlapping right eye blink. Since the signal measured by sensors is a linear combination of independent components 492 and 494, ICA separation step 425 provides independent source signals 492 and 494. As shown in the figure, multiple mixed EEG signals 405 may be analyzed in a like manner, wherein each of the EEG signals is provided by a different sensor placed on a different part of the subject's face.

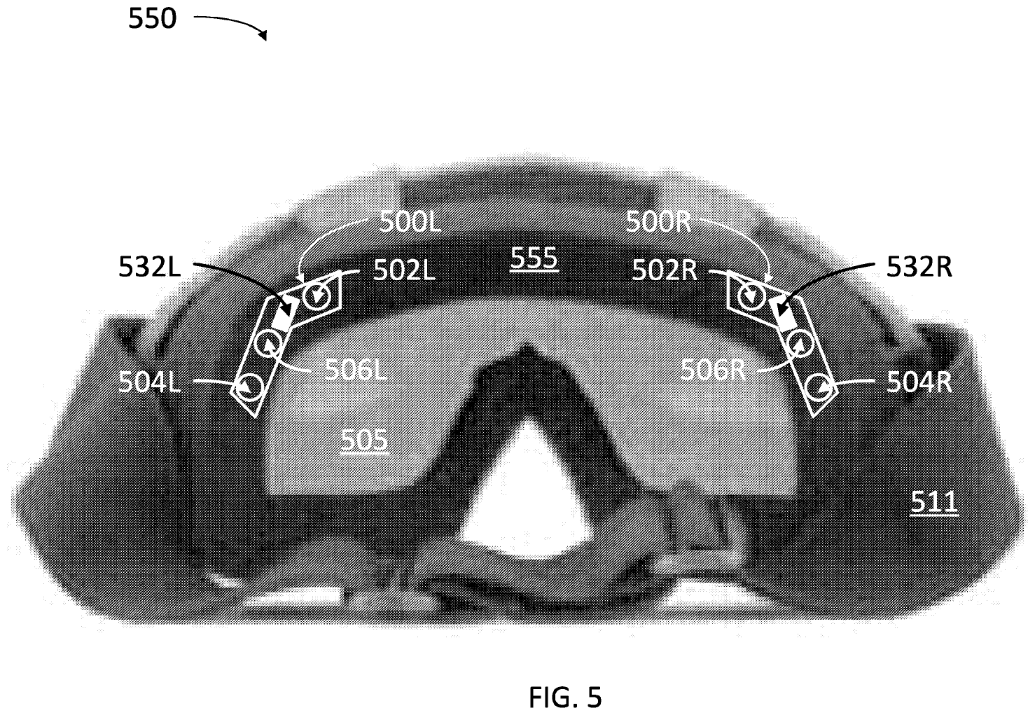

FIG. 5 provides a depiction of a headset 550 including left and right blink detectors 500L and 500R (hereinafter, collectively referred to as “blink detectors 500”), according to some embodiments. Each of blink detectors 500 includes a first sensor 502L(R), a second sensor 504L(R), and/or a third sensor 506L(R), hereinafter, collectively referred to as “sensors 502,” “sensors 504,” and “sensors 506.” Electronic components 532L and 532R (hereinafter, collectively referred to as “electronic components 532”) may include circuitry for control and processing of signals from sensors 502, 504 and 506 (cf. electronic component 132).

Blink detectors 500 are mounted on a facial interface 555 so that, when a user puts headset 550 on, sensors 502, 504, and 506 contact the user's skin and adhere to it, for proper sensing. In some embodiments, instead of left (L) and right (R) blink detectors 500, headset 550 may include only one (e.g., either 500L or 500R). The convenience of having two blink detectors 500 in headset 550 is that many of the background and other signal interferences are the same, or similar, for both eyes, and therefore a simple cancellation of the signals coming from both sensors dramatically improves the signal to noise ratio (SNR) for blink detection. In many instances, fatigue-related blinks occur simultaneously, or quasi-simultaneously, for both eyes.

Headset 550 includes an eyepiece 505, which has a display to provide augmented reality imagery to the user. The display may occupy the entire area of eyepiece 505, or at least a portion of it (e.g., toward the left eye or the right eye). Headset 550 is securely adjusted to the user's head via a strap 511.

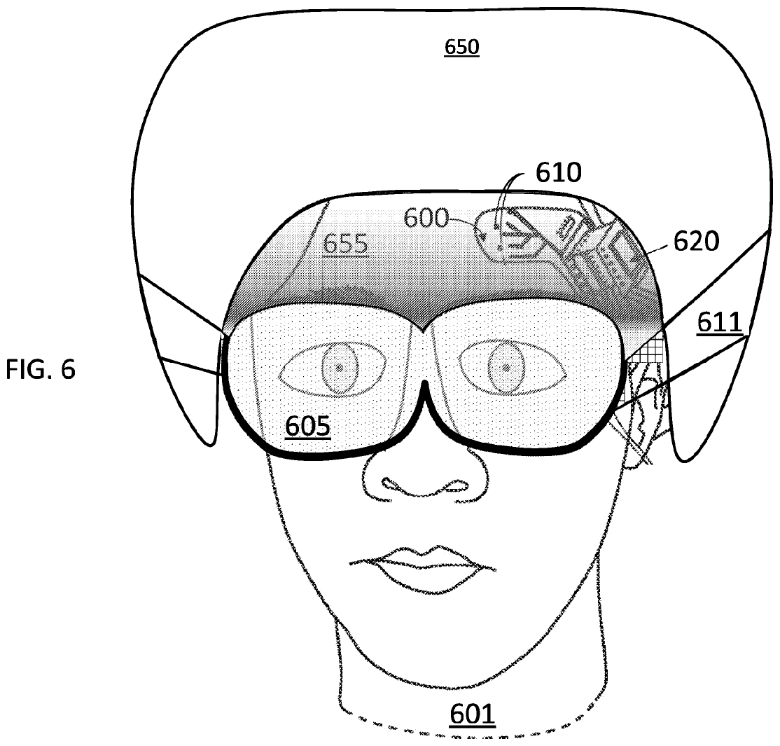

FIG. 6 illustrates a helmet 650 including a blink detector 600 for subject 601, according to some embodiments. Blink detector 600 is mounted on facial interface 655, and headset 650 is securely attached to the head of subject 601 via a strap 611. Blink detector 600 monitors subject fatigue and includes first and second sensors, hereinafter, collectively referred to as “sensors 610,” and circuitry 620. Sensors 610 are configured to sense electrical signals associated with eye blinks, as described in detail above. Sensors 610 may include one or more first and second electrodes, where the electrodes are surface electrodes that are dry electrodes. As discussed in detail above, blink detector 600, including circuitry 620 thereof, may be configured in accordance with an algorithm to detect eyelid muscle contraction based on signals from sensors 610; i.e., signals detected by sensors 610 may be subjected to an algorithm for detecting eye blinks.

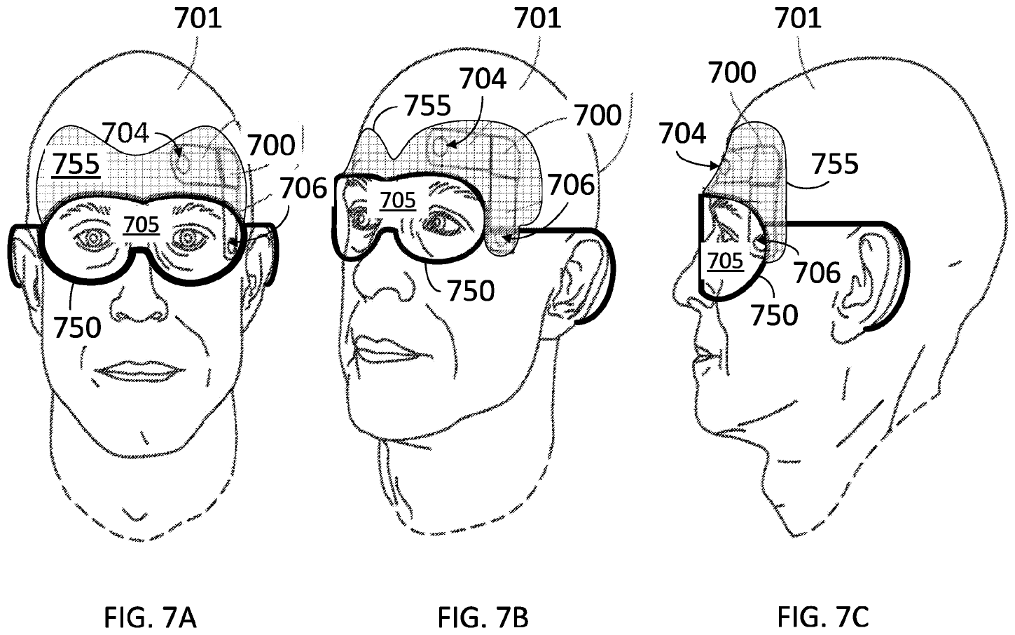

FIGS. 7A-7C illustrate different views of a headset 750 including a blink detector 700 for a user 701, according to some embodiments. A facial interface 755 supports the eyepiece 705 and blink detector 700.

Blink detector 700 includes a single patch that adheres to a facial location once the user puts headset 750 on. Blink detector 700 includes first and second sensors 704 and 706 disposed along a vertical axis and a horizontal axis, respectively.

FIG. 7A shows a view of headset 750 when subject 701 is facing forward.

FIG. 7B shows a view of headset 750 when subject 701 is facing slightly forward and slightly to the side.

FIG. 7C shows a view of headset 750 when subject 701 is facing to the side.

In an embodiment, the device or the system additionally comprises a mechanism that permits a user to activate or deactivate the device. For example, the device can include a button that a user can depress or push to active or deactivate the device. Alternatively, the software application can include an electronic button that the user can touch to activate or deactivate the device. The trigger mechanism permits a user to initiate monitoring of eye blink information and to cease monitoring of eye blink information, or to attach a label to a data set. For example, a user experiencing a symptom of, or concern regarding, fatigue or drowsiness can touch the trigger mechanism to label when the symptom or symptoms occur. The algorithm inspecting the data can look for the label to scrutinize the data in the labeled time frame to determine whether or the extent to which fatigue or drowsiness occurred. The label can take the form of an electrical spike in the data set, that is easily detected by the algorithm. The device or the system can also include an indicator to alert a user of information, such as low battery, circuit failure, on or off status. The indicator can be a light, a sound, a haptic, or the like.

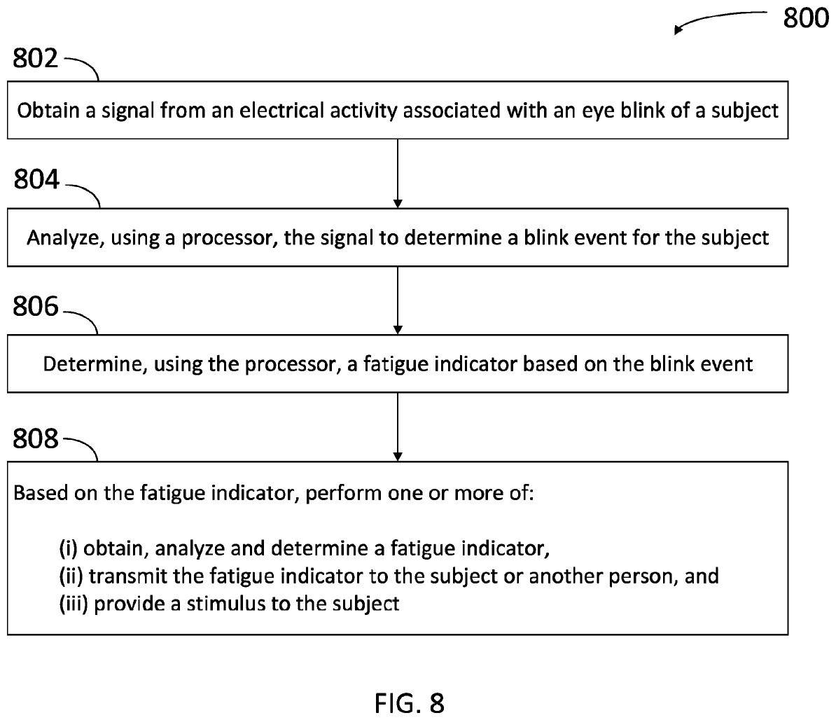

FIG. 8 is a flow chart illustrating steps in a method 800 for determining a fatigue indicator of a subject, according to some embodiments. Method 800 may include at least one or more of the steps performed by a processor executing instructions stored in a memory, as disclosed herein. In some embodiments, the instructions may include receiving signals from sensors in a blink detector, such as a blink detector mounted on a headset, as disclosed herein (e.g., sensors 102, 104, 106, 108, 302, 304, and 306, 502, 504, and 506). In some embodiments, a method consistent with the present disclosure may include at least one or more of the steps in method 800 performed in a different order, simultaneously, quasi-simultaneously, or overlapping in time.

Step 802 includes obtaining a signal from electrical activity associated with an eye blink of a subject.

Step 804 includes analyzing, using a processor, the signal to determine a blink event for the subject.

Step 806 includes determining, using the processor, a fatigue indicator based on the blink event. In certain cases, step 806 includes determining, using the processor, a fatigue indicator based on more than one blink events.

Based on the fatigue indicator determined at step 806, step 808 includes performing one or more of (i) continuing said obtaining, analyzing and determining; (ii) transmitting the fatigue indicator, e.g., information regarding the fatigue indicator, to the subject or another person; or (iii) providing a stimulus to the subject.

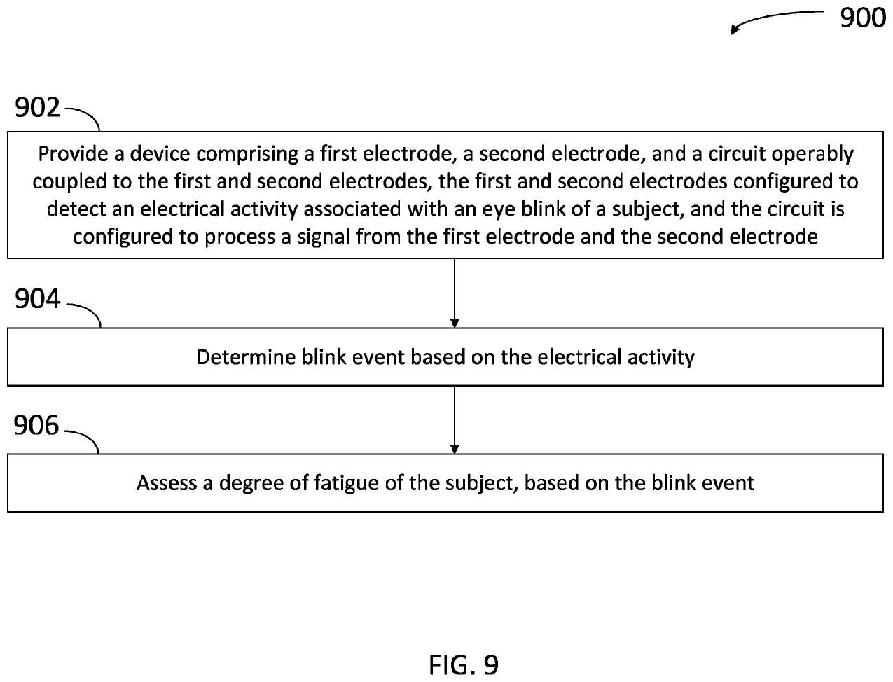

FIG. 9 is a flow chart illustrating steps in a method 900 for determining a fatigue indicator of a subject, according to some embodiments. Method 900 may include at least one or more of the steps performed by a processor executing instructions stored in a memory, as disclosed herein. In some embodiments, the instructions may include receiving signals from sensors in a blink detector, such as a blink detector mounted on a headset, as disclosed herein (e.g., sensors 102, 104, 106, 108, 302, 304, and 306, 502, 504, and 506). In some embodiments, a method consistent with the present disclosure may include at least one or more of the steps in method 900 performed in a different order, simultaneously, quasi-simultaneously, or overlapping in time.

Step 902 includes providing a device comprising a first electrode, a second electrode, and a circuit operably coupled to the first and second electrodes, the first and second electrodes configured to detect an electrical activity associated with an eye blink of a subject, and the circuit is configured to process a signal from the first electrode and the second electrode. In some embodiments, step 902 includes transmitting, with a transmitter, a signal related to the electrical activity associated with the eye blink of the subject to the circuitry or to another device, such as a mobile device. In some embodiments, step 902 includes remotely coupling the circuitry or another device, such as a mobile device, to the first and second electrodes via a wireless transmitter. In some embodiments, the device is affixed to a headset, and step 902 further includes setting the headset on the subject and verifying that the first electrode and the second electrode provide an adequate signal associated with eye blinks, such as a signal exhibiting a signal to noise ratio above a selected threshold. In some embodiments, the device or the headset further includes a sensor selected from a gyroscope, an accelerometer, an internal mass unit or a magnetometer or a photodetector, and step 902 further includes assessing the degree of fatigue of the subject based on a signal from the gyroscope, the accelerometer, the internal mass unit or the magnetometer or the photodetector, in some cases, in conjunction with a signal derived from the first and second electrodes. In some embodiments, the sensor is on the headset, and the circuitry is on the headset. In some embodiments, step 902 includes detecting electrical activity associated with an eye blink of the subject when the device is positioned near an eye of the subject, and evaluating the signal based on the electrical activity to determine a blink event. In some embodiments, step 902 includes providing an interface between the device and the subject through a software application installable on a mobile device. In some embodiments, step 902 includes fixing the device to a face of the subject such that the first electrode lies along a first axis that splits an eye of the subject vertically, and the second electrode lies along a second axis that splits the eye of the subject horizontally.

Step 904 includes determining a blink event based on the electrical activity detected by the device in step 902. In some embodiments, step 904 includes classifying, with an algorithm, the signal as a blink event or characteristics of a blink event, such as characteristics selected from a blink duration, a blink frequency, a blink amplitude, a blink velocity, and a blink burst. In some embodiments, a blink duration is the period when an eye, or both eyes are closed. In some embodiments, the blink duration is a length of time from the starting point of eyelid closure to the end point of the eyelid opening process. In some embodiments, a blink frequency is a number of blinks in a defined period. In some embodiments, a blink amplitude is an amplitude of eye opening taken as the difference in height between eyelid closure and end point of eyelid opening. In some embodiments, a blink velocity is a velocity of the eyelid(s). In some embodiments, a blink burst is a series of blinks between 0.5-2 seconds. In some embodiments, a blink burst is a burst of rapid blinking events. In some embodiments, step 904 further includes measuring a mean ocular drift velocity during a fixation of the subject on an object of interest. In some embodiments, step 904 further includes measuring a number of fixations, a mean fixation duration, and a fixation distance based on an electrical activity detected by the first electrode and the second electrode. In some embodiments, step 904 further includes measuring a micro-saccade motion of an eye of the subject, including a peak velocity-magnitude slope of the micro-saccade motion. In some embodiments, step 904 further includes measuring a saccade duration, a saccade peak velocity, a saccade peak velocity-magnitude slope, and an area under a curve of the saccade peak velocity-magnitude slope. In some embodiments, step 904 further includes measuring an eye blink count, an eye blink frequency, and a mean eye blink duration. In some embodiments, step 904 further includes identifying a blink burst when more than two eye blinks occur in a period of time less than a pre-selected threshold. In some embodiments, step 904 further includes determining a percentage of eyelid closure as a proportion of time in a minute that an eye of the subject is at least 80% closed.

Step 906 includes assessing a degree of fatigue of the subject, based on one or more blink events or other determinations made in step 904. In some embodiments, step 906 includes relaying the degree of fatigue to a processor and/or memory that is/are on a computer or a mobile device. In some embodiments, the subject is an operator of a vehicle, and step 906 includes transmitting the degree of fatigue to the vehicle, or to a vehicle command center.

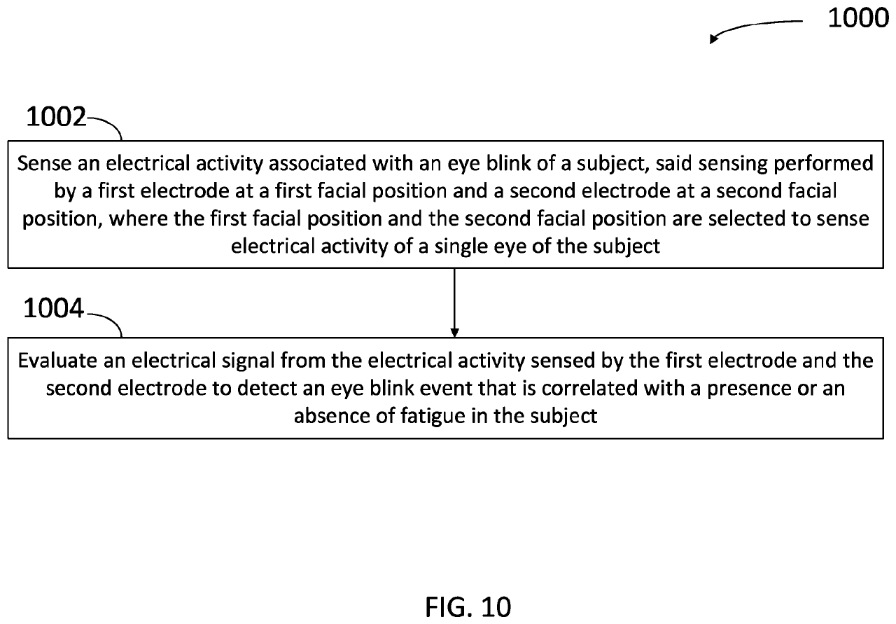

FIG. 10 is a flow chart illustrating steps in a method 1000 for determining fatigue in a subject, according to some embodiments. Method 1000 may include at least one or more of the steps performed by a processor executing instructions stored in a memory, as disclosed herein. In some embodiments, the instructions may include receiving signals from sensors in a blink detector mounted on a headset, as disclosed herein (e.g., sensors 102, 104, 106, 108, 302, 304, and 306, 502, 504, and 506). In some embodiments, a method consistent with the present disclosure may include at least one or more of the steps in method 1000 performed in a different order, simultaneously, quasi-simultaneously, or overlapping in time.

Step 1002 includes sensing electrical activity associated with an eye blink of a subject, said sensing performed by a first electrode at a first facial position and a second electrode at a second facial position, where the first facial position and the second facial position are positioned to sense electrical activity of a single eye on the subject. In some embodiments, step 1002 is performed when the subject is active, to generate an active data set. In some embodiments, step 1002 is performed when the subject is in a non-fatigued state, to generate a baseline data set. In some embodiments, step 1002 further includes sensing electrical activity associated with eye movement of the subject. In some embodiments, step 1002 further includes receiving an input related to the subject, and method 1000 uses the input and the blink event to predict or determine fatigue in the subject. In some embodiments, step 1002 further includes selecting an input from external data, such as, for example, biometric data, subject identification, subject sleep information, and subject work information. In some embodiments, step 1002 further includes selecting biometric data from one or more of sweat production, lactate production, body temperature, heart rate, and an involuntary muscle contraction.

Step 1004 includes evaluating an electrical signal from the electrical activity sensed by the first electrode and the second electrode to detect an eye blink event that is correlated with a presence or an absence of fatigue in the subject in step 1002. In some embodiments, step 1004 includes comparing the active data set, i.e., data collected from an individual subject in step 1002, to a baseline dataset. In some embodiments, step 1004 includes comparing an active data set to a control data set generated from electrical activity associated with eye blinks of a population of subjects, each subject in a non-fatigued state. In some embodiments, step 1004 further includes transmitting electrical signals from first and second electrodes configured to detect electrical activity associated with an eye blink of a subject to a processor operably connected to a memory comprising an algorithm, and said evaluating is conducted by the algorithm. In some embodiments, the algorithm evaluates the transmitted electrical signals to determine a blink event and/or characteristics thereof, such as characteristics selected from blink duration, blink frequency, blink bursts, and blink amplitude.

### Illustrative Applications of the Methods

In an embodiment, the device and/or system is used to detect whether a vehicle operator, e.g., a pilot, driver, engineer, captain, astronaut, is fatigued, so as to improve or ensure vehicle safety, including safety of the operator and any passengers. The device is disposed on or affixed to a headpiece that is worn by the operator. For example, in an embodiment, an aviation helmet includes the device as described herein, positioned on the helmet so the sensors detect eye blinks of the operator. In another example, the device is affixed to or integral with a helmet or cap worn by an astronaut, pilot, ship captain, train engineer, drone operator or motor vehicle driver. The device is affixed to the headwear such that when it is being worn by the operator, the sensors are positioned to detect eye blinks. In an embodiment, the sensors contact the skin of the subject, to detect electrical activity associated with eye blinks and/or eye movement.

By way of specific example, assessing fatigue in airplane pilots is contemplated. As can be appreciated, fatigue is a safety risk to civil and military aviation, where in-flight performance of the pilot depends on physical and psychomotor skills.

### Hardware Overview

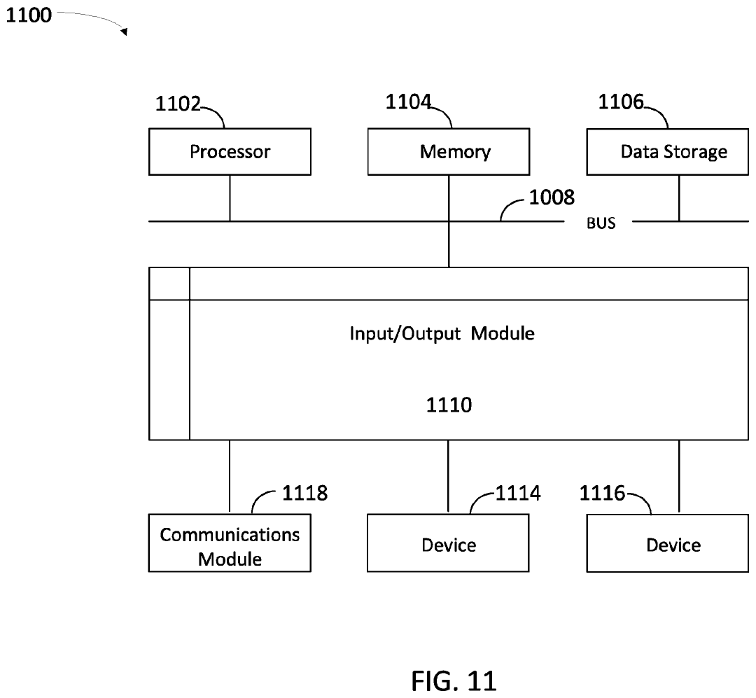

FIG. 11 is a block diagram illustrating an exemplary computer system 1100 for use with, for example, the client device 100 and/or 240 and/or circuitry thereof 130 and 230 of FIGS. 1A-1B and 2, or with which methods 800-1000 can be implemented. In certain aspects, the computer system 1100 may be implemented using hardware or a combination of software and hardware, either in a dedicated server, or integrated into another entity, or distributed across multiple entities.

Computer system 1100 includes a bus 1108 or other communication mechanism for communicating information, and a processor 1102 (e.g., processor 212) coupled with bus 1108 for processing information. By way of example, the computer system 1100 may be implemented with one or more processors 1102. Processor 1102 may be a general-purpose microprocessor, a microcontroller, a Digital Signal Processor (DSP), an Application Specific Integrated Circuit (ASIC), a Field Programmable Gate Array (FPGA), a Programmable Logic Device (PLD), a controller, a state machine, gated logic, discrete hardware components, or any other suitable entity that can perform calculations or other manipulations of information. Suitable processors may be commercially available processors available from, for example, Intel or AMD or ARM or IBM or NVidia.

Computer system 1100 can include, in addition to hardware, code that creates an execution environment for the computer program in question, e.g., code that constitutes processor firmware, a protocol stack, a database management system, an operating system, or a combination of one or more of them stored in an included memory 1104 (e.g., memory 222), such as a Random Access Memory (RAM), a flash memory, a Read-Only Memory (ROM), a Programmable Read-Only Memory (PROM), an Erasable PROM (EPROM), registers, a hard disk, a removable disk, a CD-ROM, a DVD, or any other suitable storage device, coupled with bus 1108 for storing information and instructions to be executed by processor 1102. The processor 1102 and the memory 1104 can be supplemented by, or incorporated in, special purpose logic circuitry.

The instructions may be stored in the memory 1104 and implemented in one or more computer program products, e.g., one or more modules of computer program instructions encoded on a computer-readable medium for execution by, or to control the operation of, the computer system 1100, and according to any method well known to those of skill in the art, including, but not limited to, computer languages such as data-oriented languages (e.g., SQL, dBase), system languages (e.g., C, Objective-C, C++, Assembly), architectural languages (e.g., Java, .NET), and application languages (e.g., PHP, Ruby, Perl, Python). Instructions may also be implemented in computer languages such as array languages, aspect-oriented languages, assembly languages, authoring languages, command line interface languages, compiled languages, concurrent languages, curly-bracket languages, dataflow languages, data-structured languages, declarative languages, esoteric languages, extension languages, fourth-generation languages, functional languages, interactive mode languages, interpreted languages, iterative languages, list-based languages, little languages, logic-based languages, machine languages, macro languages, metaprogramming languages, multiparadigm languages, numerical analysis, non-English-based languages, object-oriented class-based languages, object-oriented prototype-based languages, off-side rule languages, procedural languages, reflective languages, rule-based languages, scripting languages, stack-based languages, synchronous languages, syntax handling languages, visual languages, wirth languages, and xml-based languages. Memory 1104 may also be used for storing temporary variables or other intermediate information during execution of instructions to be executed by processor 1102.

A computer program as discussed herein does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, subprograms, or portions of code). A computer program can be deployed to be executed on one computer or on multiple computers that are located at one site or distributed across multiple sites and inter-coupled by a communication network. The processes and logic flows described in this specification can be performed by one or more programmable processors executing one or more computer programs to perform functions by operating on input data and generating output.

Computer system 1100 further includes a data storage device 1106 such as a magnetic disk or optical disk, coupled with bus 1108 for storing information and instructions. Computer system 1100 may be coupled via input/output module 1110 to various devices. Input/output module 1110 can be any input/output module. Exemplary input/output modules 1110 include data ports such as USB ports. The input/output module 1110 is configured to connect to a communications module 1118. Exemplary communications modules 1118 include networking interface cards, such as Ethernet cards and modems and wireless networking modules and Bluetooth modules and the like. In certain aspects, input/output module 1110 is configured to connect to a plurality of devices, such as an input device 1114 and/or an output device 1116. Exemplary input devices 1114 include a keyboard and a pointing device, e.g., a mouse or a trackball or a touchscreen, by which a user can provide input to the computer system 1100. Other kinds of input devices 1114 can be used to provide for interaction with a user as well, such as a tactile input device, visual input device, audio input device, or brain-computer interface device. For example, feedback provided to the user can be any form of sensory feedback, e.g., visual feedback, auditory feedback, or tactile feedback; and input from the user can be received in any form, including acoustic, speech, tactile, or brain wave input. Exemplary output devices 1116 include display devices, such as an LCD (liquid crystal display) monitor, for displaying information to the user.

According to one aspect of the present disclosure, a client device and/or server can be implemented, at least in part, using a computer system 1100 in response to processor 1102 executing one or more sequences of one or more instructions contained in memory 1104. Such instructions may be read into memory 1104 from another machine-readable medium, such as data storage device 1106. Execution of the sequences of instructions contained in main memory 1104 causes processor 1102 to perform the process steps described herein. One or more processors in a multi-processing arrangement may also be employed to execute the sequences of instructions contained in memory 1104. In alternative aspects, hard-wired circuitry may be used in place of or in combination with software instructions to implement various aspects of the present disclosure. Thus, aspects of the present disclosure are not limited to any specific combination of hardware circuitry and software.

Various aspects of the subject matter described in this specification can be implemented in a computing system that includes a back-end component, e.g., a data server, or that includes a middleware component, e.g., an application server, or that includes a front-end component, e.g., a client computer having a graphical consumer interface or a Web browser through which a consumer can interact with an implementation of the subject matter described in this specification, or any combination of one or more such back-end, middleware, or front-end components. The components of the system can be inter-coupled by any form or medium of digital data communication, e.g., a communication network. The communication network can include, for example, any one or more of a LAN, a WAN, the Internet, and the like. Further, the communication network can include, but is not limited to, for example, any one or more of the following network topologies, including a bus network, a star network, a ring network, a mesh network, a star-bus network, tree or hierarchical network, or the like. The communications modules can be, for example, modems or Ethernet cards.

Computer system 1100 can include clients and servers. A client and server are generally remote from each other and typically interact through a communication network. The relationship of client and server arises by virtue of computer programs running on the respective computers and having a client-server relationship to each other. Computer system 1100 can be, for example, and without limitation, a desktop computer, laptop computer, or tablet computer. Computer system 1100 can also be embedded in another device, for example, and without limitation, a mobile telephone, a PDA, a mobile audio player, a Global Positioning System (GPS) receiver, a video game console, and/or a television set top box.

The term “machine-readable storage medium” or “computer-readable medium” as used herein refers to any medium or media that participates in providing instructions to processor 1102 for execution. Such a medium may take many forms, including, but not limited to, non-volatile media, volatile media, and transmission media. Non-volatile media include, for example, optical or magnetic disks, such as data storage device 1106. Volatile media include dynamic memory, such as memory 1104. Transmission media include coaxial cables, copper wire, and fiber optics, including the wires forming bus 1108. Common forms of machine-readable media include, for example, floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH EPROM, any other memory chip or cartridge, or any other medium from which a computer can read. The machine-readable storage medium can be a machine-readable storage device, a machine-readable storage substrate, a memory device, a composition of matter affecting a machine-readable propagated signal, or a combination of one or more of them.

Many variations on the devices, systems, methods, and kits described herein will be apparent from this disclosure. For example, depending on the embodiment, certain acts, events, or functions of any of the algorithms described herein can be performed in a different sequence, can be added, merged, or left out altogether (e.g., not all described acts or events are necessary for the practice of the algorithms).

Moreover, in certain embodiments, acts or events can be performed concurrently, e.g., through multi-threaded processing, interrupt processing, or multiple processors or processor cores or on other parallel architectures, rather than sequentially. In addition, different tasks or processes can be performed by different machines and/or computing systems that can function together.

The various algorithm steps described in connection with the embodiments disclosed herein can be implemented as electronic hardware, computer software, or combinations of both. To clearly illustrate this interchangeability of hardware and software, various illustrative steps have been described above generally in terms of their functionality. Whether such functionality is implemented as hardware or software depends upon the particular application and design constraints imposed on the overall system. The described functionality can be implemented in varying ways for each particular application, but such implementation decisions should not be interpreted as causing a departure from the scope of the disclosure.

The various illustrative steps, components, and computing systems described in connection with the embodiments disclosed herein can be implemented or performed by a machine, such as a general purpose processor, a graphics processor unit, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor can include a general purpose processor, such as a microprocessor, but in the alternative, the processor can be a controller, microcontroller, or state machine, combinations of the same, or the like. A processor can also be implemented as a combination of computing devices, e.g., a combination of a DSP and a microprocessor, a plurality of microprocessors, one or more microprocessors in conjunction with a DSP core, or any other such configuration. Although described herein primarily with respect to digital technology, a processor can also include primarily analog components. A computing environment can include any type of computer system, including, but not limited to, a computer system based on a microprocessor, a graphics processor unit, a mainframe computer, a digital signal processor, a portable computing device, a personal organizer, a device controller, and a computational engine within an appliance, to name a few.

The steps of a method, process, or algorithm, and database used in said steps, described in connection with the embodiments disclosed herein can be embodied directly in hardware, in a software module executed by a processor, or in a combination of the two. A software module, engine, and associated databases can reside in memory resources such as in RAM memory, FRAM memory, flash memory, ROM memory, EPROM memory, EEPROM memory, registers, hard disk, a removable disk, a CD-ROM, or any other form of non-transitory computer-readable storage medium, media, or physical computer storage known in the art. An exemplary storage medium can be coupled to the processor such that the processor can read information from, and write information to, the storage medium. In the alternative, the storage medium can be integral to the processor. The processor and the storage medium can reside in an ASIC. The ASIC can reside in a user terminal. In the alternative, the processor and the storage medium can reside as discrete components in a user terminal.

Also provided are kits that include at least one or more wearable devices, e.g., as described above. In some instances, a kit may include the parts of the device or disparate components of a system. The kit components may be present in packaging, which packaging may be sterile, as desired.

Also present in the kit may be instructions for using the kit components. The instructions may be recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or sub-packaging), etc. In other embodiments, the instructions are present as an electronic storage data file present on a suitable computer readable storage medium, e.g., portable flash drive, DVD- or CD-ROM, etc. In other embodiments, the instructions are accessible at a given website address where they can be viewed and/or downloaded by a user. The instructions may take any form, including complete instructions for how to use the device or to troubleshoot the device.

Accordingly, in one embodiment, a kit for monitoring eye blink events of a subject comprises a wearable device for monitoring eye blink events of a subject and configured to be applied to a single side of a subject's face during use, as described herein. The device comprises first and second sensors configured to sense eye blink events of the subject; and circuitry operably coupled to the sensors and configured to detect eye blink events based on signals from the first and second sensors; and packaging for the device.

In one embodiment, the device of the kit further comprises a third sensor configured to sense head movement, position and/or orientation, wherein the circuitry is operably coupled to the third sensor and is further configured to detect head movement, position and/or orientation based on signals from the third sensor.

In one embodiment, the device of the kit further comprises a photosensor configured to sense ambient light, wherein the circuitry is operably coupled to the photosensor and is configured to detect ambient light based on signals from the photosensor.

In one embodiment, the device of the kit further comprises a storage component operably coupled to the circuitry, wherein the circuitry and the storage component are configured to record eye blink data and/or head movement, position and/or orientation data onto the storage component. In one embodiment, the circuitry is further configured to recognize one or more specific patterns of eye blink sensor data associated with fatigue or drowsiness.

In another embodiment, a kit for monitoring eye blink events of a subject comprises a wearable device for monitoring eye blink events of a subject and configured to be applied to a single side of a subject's face during use. The device comprises first and second sensors configured to sense eye blink events of the subject and a transmitter (or communications unit) configured to transmit signals sensed by the first and second sensors to remote circuitry configured to receive signals transmitted by the transmitter and to detect eye blink events based on signals from the first and second sensors, and packaging for the device.

In another embodiment, a kit for monitoring eye blink events of a subject comprises a headset comprising a wearable device for monitoring eye blink events of a subject, as described herein, and packaging for the headset. In another embodiment, a kit for monitoring eye blink events of a subject comprises a system comprising a wearable device for monitoring eye blink events of a subject, as described herein, and packaging for the system.

Based on the foregoing, it is appreciated that a wearable device is contemplated. Further based on the foregoing, it is appreciated that a portable device is contemplated. The device includes at least one sensor configured to provide output data corresponding to eye blinks by a user wearing the device, and a control logic including instructions for (i) retrieving and/or receiving or obtaining the output data from the sensor, wherein the output contains information about the user's eye blink or blinks; and (ii) decorrelation or de-mixing of the output data into data indicative of eye blink or blinks, or characteristics thereof, to create a diagnostic profile of signal; and (iii) conveying the diagnostic profile for diagnosis. In an embodiment, the output data is sensitive to head motion of the user, and the control logic includes an algorithm to account for or exclude head motion. In an embodiment, the diagnostic profile is based on signal from the wearable device processed by an algorithm as described herein.

Also as described herein, it will be appreciated that in one embodiment, the wearable device transmits a mixed signal of electrical potential data, such as, for example, corneo-retinal potential (CRP) data, to a computing device that analyzes the mixed signal to separate a signal arising from one or more eye blinks from the mixed signal data set, and using such to confirm or determine or assess fatigue or drowsiness of the subject.

Another embodiment of the system includes a wearable device and a software application, where the software application resides on a computing device and is configured to interact with the wearable device to (i) provide instructions/feedback to a user of the wearable device (e.g., loss of adherence or failure; successful transmission of eye blink data); (ii) analyze data collected from the device and/or transmission of the raw data from the wearable device or analyzed data, to a medical provider; and/or (iii) generate a report to classify fatigue or drowsiness state of the subject and/or clinically significant indicative eye blink(s) or eye blink characteristics and/or fatigue or drowsiness state of the subject. In one embodiment, a camera on the computing device is used to provide feedback about proper placement of the wearable device on the face. In an embodiment, raw or analyzed data from the system is transmitted to a centralized storage and analysis computing system.

Notwithstanding the appended claims, the disclosure is also defined by the following clauses:

1. A method for assessing fatigue of a subject, comprising:
providing a device comprising a first electrode, a second electrode, and a circuit operably coupled to the first and second electrodes, the first and second electrodes configured to detect an electrical activity associated with an eye blink of a subject, and the circuit is configured to process a signal from the first electrode and the second electrode;
measuring a blink event based on the electrical activity; and
assessing a degree of fatigue of the subject, based on the blink event.

2. The method of clause 1, further comprising transmitting, with a transmitter, a signal related to the electrical activity associated with the eye blink of the subject to the circuit.

3. The method of any one of clauses 1-2, further comprising remotely coupling the circuit to the first and second electrodes via a wireless transmitter.

4. The method of any one of clauses 1-3, wherein the device is affixed to a headset, further comprising setting the headset on the subject, and verifying that the first electrode and the second electrode provide a signal to noise ratio above a selected threshold.

5. The method of clause 4, wherein the device or the headset further comprises a sensor selected from a gyroscope, an accelerometer, an internal mass unit or a magnetometer, further comprising assessing the degree of fatigue of the subject based on a signal from the gyroscope, the accelerometer, the internal mass unit or the magnetometer.

6. The method of clause 5, wherein the sensor is on the headset, and the circuit is on the headset.

7. The method of any one of clauses 1-6, further comprising detecting electrical activity associated with an eye blink of the subject when the device is positioned near an eye of the subject, and evaluating the signal based on the electrical activity to determine a blink event.

8. The method of clause 7, further comprising relaying the signal to a processor operably connected to a memory comprising an algorithm, and said evaluating is conducted by the algorithm.

9. The method of clause 8, wherein the relaying is to a processor and/or memory that is/are on a computer or a mobile device.

10. The method of clause 9, further comprising providing an interface between the device and the subject through a software application installable on a mobile device.

11. The method of any one of clauses 8-10, wherein evaluating by the algorithm comprises classifying the signal as a blink event selected from a blink duration, a blink frequency, a blink amplitude, a blink velocity, and a blink burst.

12. The method of any one of clauses 1-11, wherein the subject is an operator of a vehicle, further comprising transmitting the degree of fatigue to the vehicle, or to a vehicle command center.

13. The method of any one of clauses 1-11, further comprising fixing the device to a face of the subject such that the first electrode lies along a first axis that splits an eye of the subject vertically, and the second electrode lies along a second axis that splits the eye of the subject horizontally.

14. The method of any one of clauses 1-11, further comprising measuring a mean ocular drift velocity during a fixation of the subject on an object of interest.

15. The method of any one of clauses 1-11, further comprising measuring a number of fixations, a mean fixation duration, and a fixation distance based on an electrical activity detected by the first electrode and the second electrode.

16. The method of any one of clauses 1-11, further comprising measuring a micro-saccade motion of an eye of the subject, including a peak velocity-magnitude slope of the micro-saccade motion.

17. The method of any one of clauses 1-11, further comprising measuring a saccade duration, a saccade peak velocity, a saccade peak velocity-magnitude slope, and an area under a curve of the saccade peak velocity-magnitude slope.

18. The method of any one of clauses 1-11, further comprising measuring an eye blink count, an eye blink frequency, and a mean eye blink duration.

19. The method of any one of clauses 1-11, further comprising identifying a blink burst when more than two eye blinks occur in a period of time less than a pre-selected threshold.

20. The method of any one of clauses 1-11, further comprising determining a percentage of eyelid closure as a proportion of time in a minute that an eye of the subject is at least 80% closed.

21. A method for predicting or determining fatigue in a subject, comprising:
sensing an electrical activity associated with an eye blink of a subject, said sensing performed by a first electrode at a first facial position and a second electrode at a second facial position, where the first facial position and the second facial position are positioned to sense electrical activity of a single eye on the subject; and
evaluating an electrical signal from the electrical activity sensed by the first electrode and the second electrode to detect an eye blink event that is correlated with a presence or an absence of fatigue in the subject.

22. The method of clause 21, wherein said sensing is performed when the subject is active, to generate an active data set.

23. The method of any one of clause 21 or clause 22, wherein said sensing is performed when the subject is in a non-fatigued state, to generate a baseline data set.

24. The method of clause 23, wherein evaluating comprises comparing the active data set to a baseline data set.

25. The method of clause 21, wherein evaluating comprises comparing an active data set to a control data set generated from electrical activity associated with eye blinks of a population of subjects, each subject in a non-fatigued state.

26. The method of any one of clauses 21-25, further comprising transmitting electrical signals from the electrical activity to a processor operably connected to a memory comprising an algorithm, and said evaluating is conducted by the algorithm.

27. The method of clause 26, wherein the algorithm evaluates the transmitted electrical signals to determine a blink event selected from blink duration, blink frequency, blink bursts, and blink amplitude.

28. The method of any one of clauses 21-27, further comprising sensing electrical activity associated with eye movement of the subject.

29. The method of any one of clauses 21-28, wherein said evaluating further comprises receiving an input related to the subject, and the algorithm uses the input and the blink event to predict or determine fatigue in the subject.

30. The method of clause 21, further comprising selecting an input from a biometric data, subject identification, subject sleep information, and subject work information.

31. The method of clause 21, further comprising selecting a biometric data from a sweat production, a lactate production, a body temperature, a heart rate, and an involuntary muscle contraction.

32. A computer-implemented method for determining fatigue of a subject, comprising:
obtaining a signal from an electrical activity associated with an eye blink of a subject;
analyzing, using a processor, the signal to determine a blink event for the subject;
determining, using the processor, a fatigue indicator based on the blink event, and
based on the fatigue indicator, performing one or more of (i) continuing said obtaining, analyzing and determining; (ii) transmitting the fatigue indicator to the subject or another person; or (iii) providing a stimulus to the subject.

33. The computer-implemented method of clause 32, wherein obtaining a signal from electrical activity comprises obtaining the signal for a period of time while the subject undertakes an action.

34. The computer-implemented method of clause 32, wherein continuing said obtaining is carried out over a period of time of least about 30 minutes.

35. The computer-implemented method of any one of clauses 32-34, wherein analyzing the signal comprises detecting a blink event based on a data set for the subject that was collected when the subject was in a non-fatigued state or based on a data set collected from a population of subjects, each in a non-fatigued state.

36. The computer-implemented method of any one of clauses 32-35, wherein the blink event is one of a blink duration, a blink frequency, a blink burst or a blink amplitude.

37. The computer-implemented method of any one of clauses 32-36, further comprising obtaining a signal from an electrical activity associated with an eye movement of the subject.

38. The computer-implemented method of clause 37, wherein the eye movement is one of a horizontal eye movement, a vertical eye movement, a torsional eye movement, a neutral gaze, a leftward gaze, a rightward gaze, an upward gaze, and a downward gaze.

39. The computer-implemented method of clauses 37-38, further comprising triggering the eye movement with an inducer of smooth pursuit, saccades or opticokinetic eye movement.

40. The computer-implemented method of any one of clauses 32-39, further comprising obtaining data related to a head movement, a position, and an orientation of the subject.

41. The computer-implemented method of any one of clauses 32-40, wherein obtaining a signal comprises obtaining a signal from a first electrode and a second electrode that are affixed to a headset used by the subject.

42. The computer-implemented method of clause 32, wherein the signal is provided by an electrode mounted on a headset, which is one of a helmet, a hat, or a goggle, further comprising verifying that the signal from an electrical activity is above a pre-selected threshold when the subject puts the headset on.

43. The computer-implemented method of any one of clauses 32-42, wherein analyzing the signal and determining a fatigue indicator comprise executing, by a processor in a computer or a mobile device, one or more instructions stored in a memory of the computer or the mobile device.

44. The computer-implemented method of clause 32, further comprising receiving a command from the subject via a software application in a mobile device that drives an interface for the subject.

45. The computer-implemented method of any one of clauses 32-44, wherein analyzing the signal comprises analyzing a signal corresponding to an eye blink and a signal corresponding to a physiological parameter of the subject, to determine the fatigue indicator.

46. A method to assess fatigue in real-time, comprising:
providing to a subject a system comprised of (i) a wearable device comprising a first electrode, a second electrode and circuitry operably connected to the first and second electrodes, the first and second electrodes configured to detect electrical activity associated with an eye blink of a subject, and the circuitry is configured to process a signal from the first and second electrodes and (ii) a software application comprising an algorithm for evaluating the signal;
instructing the subject to (i) install the software application on a computing device, if needed, (ii) place the wearable device on their person proximal to an eye, and (iii) using the software application on the computing device, initiate a baseline assessment of eye movement and eye blink; and
instructing the subject to begin a planned activity and to continue to wear the wearable device during the electrical activity, wherein the wearable device senses electrical activity associated with eye blink, transmits signal corresponding to the electrical activity to a processor on the computing device for processing by an algorithm in the software application, to determine a blink event to assess fatigue.

47. A headset, comprising:
an eyepiece configured to provide an image to a user;
a facial interface configured to fix the eyepiece to face of the user;
and a blink detector mounted on the facial interface, the blink detector comprising:
a first electrode and a second electrode mounted on the facial interface and configured to contact a skin of the user to detect an electrical activity associated with an eye blink of the user; and
a circuit operably coupled to the first and second electrodes, configured to process a signal from the first and the second electrodes and identify a blink event by the user based on the electrical activity.

48. The headset of clause 47, wherein the first electrode and the second electrode are mounted on the facial interface such that one of the first electrode or the second electrode lies along an axis that splits an eye of the user vertically.

49. The headset of clause 47, wherein the first electrode and the second electrode are mounted on the facial interface such that one of the first electrode or the second electrode lies along an axis that splits an eye of the user horizontally.

50. The headset of clause 47, wherein the blink detector further includes a third electrode that contacts the skin of the user at a point separated from the first electrode and the second electrode and is configured to detect a third signal of the electrical activity associated with the eye blink of the user; and wherein the circuit enhances a sensitivity to the blink event based on the third signal of the electrical activity.

51. The headset of clause 47, wherein the blink detector further includes a third electrode that contacts the skin of the user at a point separated from the first electrode and the second electrode and is configured to detect a third signal of the electrical activity associated with the eye blink of the user, and wherein the circuit identifies the blink event with a larger spatial tolerance for a location of the first electrode, the second electrode, and the third electrode.

52. A kit comprising: a device according to any of clauses 1 to 20; and packaging for the device.

53. A kit comprising: a system according to clause 46; and packaging for the system.

54. A kit comprising: a headset according to any of clauses 47 to 51;
and packaging for the headset.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of the present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112(f) or 35 U.S.C. § 112(6) is not invoked.

1. A method for assessing fatigue of a subject, comprising:
providing a device comprising a first electrode, a second electrode, and a circuit operably coupled to the first and second electrodes, the first and second electrodes configured to detect an electrical activity associated with an eye blink of a subject, and the circuit is configured to process a signal from the first electrode and the second electrode;
measuring a blink event based on the electrical activity; and
assessing a degree of fatigue of the subject, based on the blink event.

2. The method of claim 1, further comprising transmitting, with a transmitter, a signal related to the electrical activity associated with the eye blink of the subject to the circuit.

3. The method of claim 1, further comprising remotely coupling the circuit to the first and second electrodes via a wireless transmitter.

4. The method of claim 1, wherein the device is affixed to a headset, further comprising setting the headset on the subject, and verifying that the first electrode and the second electrode provide a signal to noise ratio above a selected threshold.

5. The method of claim 4, wherein the device or the headset further comprises a sensor selected from a gyroscope, an accelerometer, an internal mass unit or a magnetometer, further comprising assessing the degree of fatigue of the subject based on a signal from the gyroscope, the accelerometer, the internal mass unit or the magnetometer.

6. The method of claim 5, wherein the sensor is on the headset, and the circuit is on the headset.

7. The method of claim 1, further comprising detecting electrical activity associated with an eye blink of the subject when the device is positioned near an eye of the subject, and evaluating the signal based on the electrical activity to determine a blink event.

8. The method of claim 7, further comprising relaying the signal to a processor operably connected to a memory comprising an algorithm, and said evaluating is conducted by the algorithm.

9. The method of claim 8, wherein the relaying is to a processor and/or memory that is/are on a computer or a mobile device.

10. The method of claim 8, wherein evaluating by the algorithm comprises classifying the signal as a blink event exhibiting a characteristic selected from a blink duration, a blink frequency, a blink amplitude, a blink velocity, and a blink burst.

11. The method of claim 1, wherein the subject is an operator of a vehicle, further comprising transmitting the degree of fatigue to the vehicle, or to a vehicle command center.

12. The method of claim 1, further comprising fixing the device to a face of the subject such that the first electrode lies along a first axis that splits an eye of the subject vertically, and the second electrode lies along a second axis that splits the eye of the subject horizontally.

13. The method of claim 1, further comprising measuring an eye blink count, an eye blink frequency, and a mean eye blink duration.

14. The method of claim 1, further comprising identifying a blink burst when more than two eye blinks occur in a period of time less than a pre-selected threshold.

15. The method of claim 1, further comprising determining a percentage of eyelid closure as a proportion of time in a minute that an eye of the subject is at least 80% closed.
